(19) United States
(12) Patent Application Publication
(10) Pub. No.: US 2009/0110727 A1
(43) Pub. Date: Apr. 30, 2009

(54) Extended release compositions of proton pump inhibitors

(75) Inventor: John Paul Carter, North Andover, MA (US)

Correspondence Address: Lahive & Cockfield, LLP/EISAI;Floor 30, Suite 3000, One Post Office Square, Boston, MA, 02109, US

(73) Assignee: Eisai R & D management Co., Ltd., Tokyo (JP)

(21) Appl. No.: 11/665,032

(22) PCT Filed: Oct. 12, 2005
(86) PCT No.: PCT/US05/36672

§ 371 Date: Jan. 14, 2009

Related U.S. Application Data
(60) Provisional application No. 60/617,165, filed on Oct. 12, 2004.

(51) Int. Cl. A61K 9/22; A61K 31/4439; A61P 1/00

(52) U.S. Cl. 424/468; 514/338

(57) ABSTRACT

The invention provides extended release compositions comprising at least one proton pump inhibitor. The invention also provides methods for treating gastrointestinal disorders by administering the compositions of the invention to patients in need of gastrointestinal therapy.

### FIELD OF THE INVENTION

The invention provides safe and effective extended release compositions comprising proton pump inhibitors and methods for treating gastrointestinal disorders with the extended release compositions.

### BACKGROUND OF THE INVENTION

Gastroesophageal reflux disease (GERD) is a common medical illness. Gastroesophageal reflux occurs when the contents of the stomach, including acids and digestive fluids, leak back past the lower esophageal sphincter into the esophagus. This produces the sensation commonly referred to as “heartburn.” Over prolonged periods, GERD can seriously compromise a patient's health. Currently, there are four options available for treatment of GERD. These options are medication, life-style modification, surgery, and/or endoscopic fundoplication. Medication is the most common treatment. Depending upon the degree of severity, a physician may prescribe medications ranging from histamine-2 receptor antagonists to proton pump inhibitors (e.g., ACIPHEX® by Eisai, Inc.). The results from treatment by medication are satisfactory in the majority of patients. Some patients, however, experience nocturnal acid breakthrough or occasional acid breakthrough. Nocturnal acid breakthrough occurs during the night, while occasional acid breakthrough can occur during the day or night.

There is a need in the art for new and improved pharmaceutical formulations that can safely and effectively treat GERD and prevent nocturnal acid breakthrough or occasional acid breakthrough. The invention is directed to this, as well as other, important ends.

### SUMMARY OF THE INVENTION

The invention provides an extended release formulation comprising at least one proton pump inhibitor and at least one polymer and, optionally, at least one hydrogel.

The invention provides an extended release formulation comprising at least one proton pump inhibitor and at least one hydrogel and, optionally, at least one polymer.

The invention provides extended release dosage forms comprising a semipermeable wall that forms a compartment comprising a composition which comprises at least one proton pump inhibitor and, optionally, at least one osmotically effective solute. The semipermeable wall comprises at least one passageway.

The invention provides extended release dosage forms comprising (1) a drug composition that comprises at least one proton pump inhibitor and a pharmaceutically acceptable carrier; (2) a first coat that surrounds the drug composition, wherein the first coat comprises at least one polymer; (3) a second coat that surrounds the first coat, the second coat comprising a composition that is permeable to the passage of fluid and impermeable to the passage of drug; and (4) an exit passageway in the first and second coats for releasing the proton pump inhibitor from the dosage form over an extended time. In one embodiment, the first coat comprises ethyl cellulose, hydroxylalkylcellulose, or a mixture thereof. In other embodiments, the dosage form further comprises an expandable composition that possesses a higher molecular weight than the pharmaceutically acceptable carrier in the drug composition.

The invention provides proton pump inhibitor dosage forms comprising (a) a therapeutically effective amount of at least one proton pump inhibitor; (b) a polymer matrix; and, optionally, (c) at least one band of an insoluble material circumscribing a portion of the surface of the polymer matrix. In one embodiment, the proton pump inhibitor is dispersed or dissolved in the polymer matrix. In other embodiments, the polymer matrix comprises at least one water-soluble polymer and at least one hydroattractant.

The invention provides dosage forms for delivering proton pump inhibitor compositions to an environment of use, wherein the dosage form comprises (a) a wall comprising a composition that is permeable to the passage of fluid and is substantially impermeable to the passage of proton pump inhibitor, which wall surrounds and forms; (b) a compartment comprising (i) a drug composition comprising at least one proton pump inhibitor; at least one osmopolymer; and, optionally, at least one osmagent; and (ii) a push composition in contact with the drug composition in the compartment, which push composition, in the presence of fluid that enters the dosage form, increases in dimension and pushes the drug composition from the dosage form; and (d) at least one exit means in the wall for delivering the drug composition from the dosage form at a controlled rate over a period of time.

The invention provides osmotic dosage forms for the extended delivery of a proton pump inhibitor to an environment of use, the osmotic dosage form comprising: (a) a wall comprising a composition that is permeable to the passage of an exterior fluid present in the environment of use and substantially impermeable to the passage of proton pump inhibitor, the wall surrounding and forming: (b) a compartment comprising (i) a first composition comprising at least one proton pump inhibitor, an osmopolymer that exhibits an osmotic pressure gradient across the wall against an external fluid, and, optionally, an osmagent that exhibits an osmotic pressure gradient across the wall against an external fluid; and (ii) a second composition comprising an osmopolymer that exhibits an osmotic pressure gradient across the wall against an external fluid, and, optionally, an osmagent that exhibits an osmotic pressure gradient across the wall against an external fluid; and (e) at least one passageway in the wall communicating with the first composition and the exterior of the dosage form for delivering the proton pump inhibitor through the passageway from the dosage form. In one embodiment, the first composition is in the compartment as a layer, and the second composition is in the compartment as a separate layer. In another embodiment, the first composition imbibes external fluid through the wall into the compartment, and the second composition imbibes external fluid through the wall into the compartment. In another embodiment, the osmopolymer comprising the second composition has a molecular weight greater than the molecular weight of the osmopolymer comprising the first composition.

The invention provides osmotic dosage forms for the extended delivery of a proton pump inhibitor formulation to an environment of use, comprising: (a) a shaped wall permeable to the passage of an exterior biological fluid and substantially impermeable to the passage of proton pump inhibitor formulation, which wall surrounds and forms: (b) a compartment comprising: (1) a proton pump inhibitor formulation, which formulation comprises at least one proton pump inhibitor, an osmotically effective solute that is soluble in the exterior fluid and exhibits an osmotic pressure gradient across the wall against the fluid and a polymer that imbibes fluid and absorbs fluid that enters the compartment; and (2) a delivery formulation, which formulation comprises an osmotically effective solute that is soluble in the exterior fluid and exhibits an osmotic pressure gradient across the wall against the fluid and a polymer that imbibes fluid and absorbs fluid that enters the compartment; and (c) at least one passageway in the wall connecting the exterior of the dosage form with the proton pump inhibitor formulation for delivering the proton pump inhibitor formulation from the dosage form to the environment at a controlled rate over a prolonged period of time.

The invention provides compositions comprising in combination: (1) a first composition comprising at least one proton pump inhibitor, an osmopolymer, and, optionally, an osmagent; and (2) a second composition in laminar arrangement with the first composition (1), which second composition (2) comprises an osmopolymer and, optionally, an osmagent; wherein compositions (1) and (2) exhibit an osmotic pressure gradient across a semipermeable polymeric film against fluid, such as aqueous and biological fluids.

The invention provides osmotic delivery systems for the extended release of at least one proton pump inhibitor to an environment of use, wherein the system comprises: (a) a shaped semipermeable wall permeable to the passage of fluid and substantially impermeable to the passage of the proton pump inhibitor, wherein the semi-permeable wall surrounds and forms; (b) a compartment comprising at least one proton pump inhibitor; and (c) an osmotic passageway in the wall communicating with the compartment and the exterior of the system for releasing the proton pump inhibitor through the osmotic passageway from the dosage form to the environment of use over time. The proton pump inhibitor is preferably soluble in an external fluid imbibed through the semipermeable wall into the compartment and exhibits an osmotic pressure gradient across the semipermeable wall against the external fluid. In other embodiments, the semipermeable wall can comprise an organic solvent soluble polymer and/or a permeability enhancer. In other embodiments, the semipermeable wall comprises an organic solvent soluble polymer and a blend of water soluble polymers which, on the application of energy used to coat the semipermeable wall of the delivery system, form a hydrophilic and substantially fluid insoluble polymer in the semipermeable wall on the dosage form. In other embodiments, the semipermeable wall comprises an organic solvent soluble polymer, a polyhydroxy polymer and a polycarboxy polymer, which polyhydroxy polymer and polycarboxy polymer react while coating the wall onto the dosage form to form a hydrophilic fluid permeability enhancing polymer blended within the organic solvent soluble polymer.

The compositions, dosage forms and systems of the invention may further comprise a partial or complete outer enteric coating layer in order to provide for release of the proton pump inhibitor into the alkaline portion of the gastrointestinal tract (e.g., small intestine, large intestine, and the like). Acid-labile proton pump inhibitors must be protected from the acidity in the gastric environment to prevent their degradation.

The compositions, dosage forms and systems of the invention may further comprise one or more enteric polymers over and/or in the passageways described herein to prevent the release of the proton pump inhibitor into the stomach, and to allow release of the proton pump inhibitor after the compositions/dosage forms/systems enter into the alkaline portion of the gastrointestinal tract (e.g., small intestine, large intestine, and the like). In this embodiment of the invention, the compositions, dosage forms and systems may optionally comprise a partial or complete enteric coating on portions other than the passageways.

The extended release compositions and dosage forms of the invention comprising proton pump inhibitors can be used to treat gastrointestinal disorders in patients in need thereof.

The invention is described in more detail below.

### BRIEF DESCRIPTION OF THE FIGURES

FIG. 1 is a general view of a dosage form provided by this invention designed, shaped and adapted for the oral administration of at least one proton pump inhibitor at a controlled rate over an extended time to a human in need of drug therapy.

FIG. 2 is a general view of the dosage form of FIG. 1, in opened section, depicting a dosage form of this invention comprising an internally housed, pharmaceutically-acceptable therapeutic drug composition comprising at least one proton pump inhibitor.

FIG. 3 is an opened view of drawing FIG. 1, illustrating a dosage form comprising a drug composition of at least one proton pump inhibitor and a separate, but initially contacting push-displacement composition comprising means for pushing the drug composition from the dosage form.

FIGS. 4A and 4B illustrate one embodiment of the delivery dosage form of the invention, the dosage form in FIG. 4A representing the active agent formulation matrix not including the insoluble material or band and the dosage form in FIG. 4B representing the banded dosage form in prepared form prior to placement in the stomach.

FIG. 5 illustrates the dosage form of FIG. 4B in its initially-swollen state after having expanded in the stomach.

FIGS. 6A and 6B illustrate the dosage form of FIG. 5 at later stages where the dosage form has eroded in the fluid environment of use.

FIGS. 7A-7D illustrate another embodiment of the invention in which two bands of insoluble material have been incorporated on the dosage form illustrated in FIG. 4A.

FIG. 8 illustrates another embodiment of the invention that incorporates a swellable polymer matrix tube or ring formed about a separate active agent reservoir for dispensing active agent to the environment of use.

FIG. 9 illustrates an embodiment of the invention that includes a band of insoluble material circumscribing a portion of the dosage form of FIG. 8.

FIG. 10 illustrates still another embodiment of the invention where the polymer matrix tube or ring is formed with split ends which in its swollen state results in the ends of the polymer tube or ring flaring outwardly and swelling to provide a larger effective diameter.

FIG. 11 is an isometric view of a delivery dosage form designed for orally administering a proton pump inhibitor to the gastrointestinal tract.

FIG. 12 is an opened view of the dosage form of FIG. 11 illustrating the structure of the dosage form of FIG. 11.

FIG. 13 is an opened view of the dosage form of FIG. 11 illustrating the dosage form in operation and delivering a proton pump inhibitor from the dosage form.

FIG. 14 is an opened view of the dosage form of FIG. 11 considered with FIG. 13 illustrating the dosage form in operation and comprising more than one passageway for delivering a major amount of a proton pump inhibitor from the dosage form.

FIG. 15 is a graph showing the weight gain as a function of time for a polymer encapsulated in a semipermeable membrane when the encapsulated polymer is placed in water.

FIG. 16 is a view of an osmotic dosage form designed for orally administering a proton pump inhibitor.

FIG. 17 is a view of the osmotic dosage form of FIG. 16 seen in opened-section for illustrating the structure of the dosage form including the wall and the compartment.

FIG. 18 is an illustration of a dosage form of the invention with a portion of the wall removed to illustrate the general structure of the dosage form.

FIG. 19 is a perspective, top view of a dosage form of the invention illustrating as one embodiment of the invention a dosage form manufactured as an oral dosage form.

FIG. 20 is an enlarged cross-sectional view of the dosage form of FIG. 19 through 3-3 depicting two walls with their interior peripheral surfaces in intimate contact with the surfaces of a wall positioned between the two walls.

FIG. 21 is an osmotic dosage form designed for orally administering a proton pump inhibitor.

FIG. 22 is an opened view of the osmotic dosage form of FIG. 21 illustrating the internal structure of the dosage form of FIG. 21.

FIGS. 23 through 25 are seen in opened section illustrating the osmotic dosage form of FIGS. 21 and 22 in operation and delivering the proton pump inhibitor from the dosage form.

FIG. 26 is an osmotic dosage form designed for orally administering a proton pump inhibitor.

FIGS. 27 through 31 are side views, partially broken away, of the osmotic dosage form of FIG. 26 illustrating the compartments of the system separated by an integrally formed contiguous expandable film.

FIG. 32 is an osmotic dosage form in cross-section showing a dosage form having two integrally formed compartments.

FIG. 33 is an osmotic dosage form similar to FIG. 32 in cross-section illustrating a dosage form embracing another design and shape according to the invention.

FIG. 34 is a view of an extended release dosage form designed and shaped for oral administration of a proton pump inhibitor.

FIG. 35 is an opened view of the dosage form of FIG. 34, which FIG. 35 illustrates the internal structure of the dosage form comprising two spaces separated by a partition, with one space containing a means for increasing the fraction of agent delivered at zero order over time by maintaining the saturated state of proton pump inhibitor in the integral unit manufactured as a dosage form.

FIG. 36 is the dosage form of FIG. 34 with a section removed for depicting the internal structure of the dosage form manufactured with a partition, a different external wall structure, an agent housing space and a contacting expanding means for increasing the fraction of proton pump inhibitor present in a saturated state in the dosage form.

FIG. 37 shows a dosage form of the invention having an enteric coating layer over the entire dosage form and, optionally, in the passageways.

FIG. 38 shows a dosage form of the invention having an enteric coating layer over the passageways, but not over the entire dosage form.

FIG. 39 shows a dosage form of the invention where enteric polymers are used to completely fill the passageways to prevent release of the proton pump inhibitor in the stomach.

FIG. 40 shows a dosage form of the invention having an enteric coating layer over the passageways and partially within the passageways of the dosage form.

FIG. 41 shows a dosage form of the invention where enteric polymers are used to partially fill the passageways to prevent release of the proton pump inhibitor in the stomach.

### DETAILED DESCRIPTION OF THE INVENTION

“Extended release” denotes the delivery of the proton pump inhibitor for up to twenty-four hours; from about 4 hours to about 24 hours; from about 4 hours to about 12 hours; from about 8 hours to about 12 hours; or from about 8 hours to about 16 hours. The extended release compositions/dosage forms of the invention can also be zero-order release compositions/dosage forms. Zero-order release denotes compositions/dosage forms that deliver the proton pump inhibitor at a uniform rate to dampen the peaks and valleys observed in non-zero order method of drug delivery.

The invention provides extended release compositions comprising at least one proton pump inhibitor and at least one polymer. The composition may optionally have an enteric coating layer. The proton pump inhibitor can be any known in the art as described herein. The enteric coating can be any known in the art as described herein. The polymer can be any known in the art. Exemplary polymers include one or more of the following:

(a) Hydrophilic polymers, such as gums (e.g., xanthan gum, locust bean gum, dextran, gellan gum, welan gum, rhamsan gum, tragacanth, pectins, acacia, karaya, alginates, agar, guar, hydroxypropyl guar, carrageenan), cellulose ethers (e.g., hydroxyalkylcelluloses and carboxyalkylcelluloses), acrylic resins and protein derived materials. The extended release compositions may contain between 1% and 80% by weight of at least one hydrophilic polymer.

(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. The hydrocarbons may having a melting point between 25° C. and 90° C. The extended release compositions may contain up to 60% by weight of at least one digestible, long chain hydrocarbon, such as a fatty (aliphatic) alcohol.

(c) Polyalkylene glycols. The extended release compositions may contain up to 60% by weight of at least one polyalkylene glycol.

(d) Other polymers described in more detail herein.

(e) Hydrogels described in more detail herein.

One extended release composition comprises at least one water soluble hydroxyalkyl cellulose, at least one C₁₂-C₃₆, preferably C₁₄-C₂₂, aliphatic alcohol and, optionally, at least one polyalkylene glycol. The at least one hydroxyalkyl cellulose is preferably a hydroxy (C₁-C₆) alkyl cellulose, such as hydroxypropylcellulose, hydroxypropylmethylcellulose and, especially, hydroxyethyl cellulose. The amount of the at least one hydroxyalkyl cellulose in the composition will be determined by the rate of proton pump inhibitor release required. Preferably, the extended release compositions contain between 1% and 25%, especially between 5% and 15% by weight of the at least one hydroxyalkyl cellulose.

The at least one aliphatic alcohol may be, for example, lauryl alcohol, myristyl alcohol, stearyl alcohol or mixtures of two or more thereof. In other embodiments, the at least one aliphatic alcohol is cetyl alcohol, cetostearyl alcohol or a mixture thereof. The amount of the at least one aliphatic alcohol in the extended release composition will be determined by the rate of proton pump inhibitor release required. It will also depend on whether at least one polyalkylene glycol is present in or absent from the composition. In the absence of at least one polyalkylene glycol, the composition preferably contains between 20% and 50% by weight of the at least one aliphatic alcohol. When at least one polyalkylene glycol is present in the composition then the combined weight of the at least one aliphatic alcohol and the at least one polyalkylene glycol preferably constitutes between 20% and 50% by weight of the total composition.

In one embodiment, the extended release compositions comprise from about 5 to about 25% acrylic resin and from about 8 to about 40% by weight aliphatic alcohol by weight of the total composition. A preferred acrylic resin comprises EUDRAGIT® RS PM.

In the composition, the ratio of, e.g., the at least one hydroxyalkyl cellulose or acrylic resin to the at least one aliphatic alcohol/polyalkylene glycol, determines, to a considerable extent, the release rate of the proton pump inhibitor from the composition. A ratio of the at least one hydroxyalkyl cellulose to the at least one aliphatic alcohol/polyalkylene glycol may be between 1:2 and 1:4; or may be a ratio of between 1:3 and 1:4.

The at least one polyalkylene glycol may be, for example, polypropylene glycol or polyethylene glycol. The number average molecular weight of the at least one polyalkylene glycol may be between 1000 and 15000; or between 1500 and 12000.

Another suitable extended release composition would comprise an alkylcellulose (especially ethyl cellulose), a C₁₂ to C₃₆ aliphatic alcohol and, optionally, a polyalkylene glycol.

In addition to the above ingredients, extended release compositions may also contain suitable quantities of other materials, e.g. diluents, lubricants, binders, granulating aids, colorants, flavorants and glidants that are conventional in the pharmaceutical art.

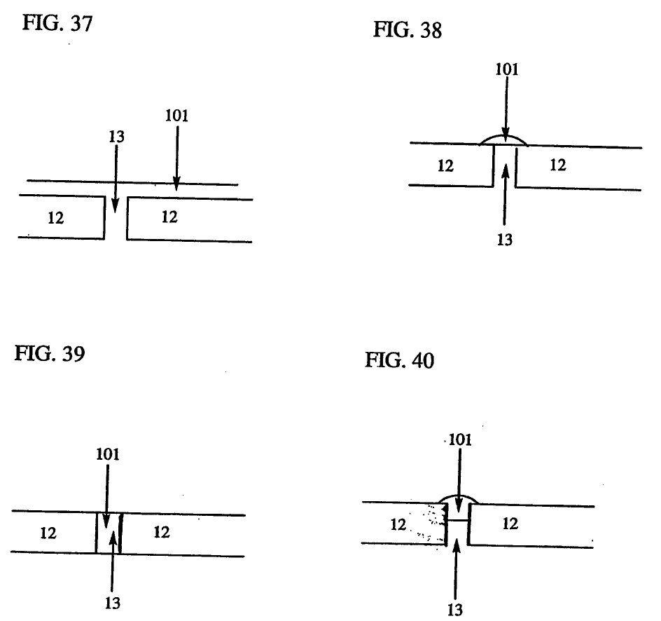

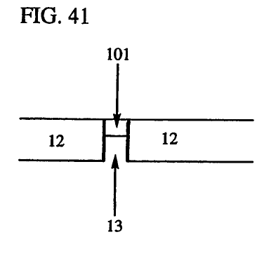

The dosage forms of the invention may comprise an enteric coating to ensure that the dosage form passes through the acidic stomach and into the alkaline small intestine. It is known that stomach acid will degrade acid-labile proton pump inhibitors. The enteric coating may comprise any polymer known in the art that is useful for forming an enteric coating and that provides for release of the proton pump inhibitor in the alkaline environment of the small intestine. As exemplified in FIG. 37, the dosage forms of the invention may comprise an enteric coating layer 101 that completely or partially covers the outer wall 12 of the dosage form. As exemplified in FIG. 38, the dosage forms of the invention may comprise an enteric coating 101 only over the passageway(s) 13 to ensure that the dosage form passes through the acidic stomach and into the alkaline small intestine before releasing the proton pump inhibitor. As exemplified in FIG. 39, the dosage forms of the invention may comprise one or more enteric polymers 101 completely filling the passageways 13 to ensure that the dosage form passes through the acidic stomach and into the alkaline small intestine. As exemplified in FIG. 40, the dosage forms of the invention may comprise one or more enteric polymers 101 that cover the passageways and partially or completely fill the passageways 13. As exemplified in FIG. 41, the dosage forms of the invention may comprise one or more enteric polymers 101 partially filling the passageways 13 to ensure that the dosage form passes through the acidic stomach and into the alkaline small intestine. In still other embodiments, the dosage forms of the invention may comprise an enteric coating beneath the outer wall to ensure that the dosage form passes through the acidic stomach and into the alkaline small intestine. One skilled in the art will appreciate that with reference to all of the dosage forms, compositions or systems described herein, an enteric coating and/or enteric polymers serve the function of preventing the release of the proton pump inhibitor in the stomach, and to allow for the release of the proton pump inhibitor in the alkaline environment of the small intestine. The enteric coating and/or enteric polymers can be used in any manner that achieves this goal, with FIGS. 37-40 being but four examples of ways to achieve this goal.

Exemplary enteric polymers include esters of cellulose and/or its derivatives (e.g., cellulose acetate phthalate, hydroxypropyl methylcellulose phthalate, hydroxypropyl methylcellulose acetate succinate), polyvinyl acetate phthalate, carboxymethylethylcellulose, acrylic acid polymers, methacrylic acid copolymers, methacrylic acid-methacrylate copolymers, shellac and mixtures of two or more thereof. These enteric polymers may be used as a dry powder or an aqueous dispersion. Some commercially available enteric polymers that may be used are methacrylic acid copolymers sold under the trademark EUDRAGIT® (L100, S100, L30D) manufactured by Rhom Pharma, CELLACEFATE® (cellulose acetate phthalate) from Eastman Chemical Co., AQUATERIC® (cellulose acetate phthalate aqueous dispersion) from FMC Corp. and AQUOT® (hydroxypropyl methylcellulose acetate succinate aqueous dispersion) from Shin Etsu.

The enteric coating may optionally further comprise at least one plasticizer and/or at least one water insoluble polymer. The plasticizer can be any known in the art. Exemplary plasticizers include triacetin, tributyl citrate, triethyl citrate, acetyl tri-butyl citrate diethyl phthalate, castor oil, dibutyl sebacate, acetylated monoglycerides, monoacetylated glycerides, diacetylated glycerides, diacetylated monoglycerides, and mixtures of two or more thereof. In one embodiment, the plasticizer is acetylated monoglycerides, monoacetylated glycerides, diacetylated glycerides, diacetylated monoglycerides, or mixtures of two or more thereof. In another embodiment, the plasticizer is diacetylated monoglycerides. The plasticizer may be present in an amount of about 3 to about 30 weight %; or from about 10 to about 25 weight % based on the weight of the enteric polymer. Any water insoluble polymer known in the art can be used. Exemplary water insoluble polymers include cellulose derivatives (e.g. ethylcellulose), polyvinyl acetate (KOLLICOAT® SR30D from BASF), neutral copolymers based on ethyl acrylate and/or methylmethacrylate, and copolymers of acrylic and methacrylic acid esters with quaternary ammonium groups, such as EUDRAGIT® NE, RS or RS30D, RL or RL30D, and the like. In one embodiment, the water insoluble polymer is ethylcellulose. The enteric polymer and water insoluble polymer may be present at a thickness of about 5 to about 60 weight %; or from about 10 to about 50 weight %. The ratio of water insoluble polymer to enteric polymer may vary from about 10:1 to about 1:2; from about 2:1 to about 1:1; or from about 4:1 to about 1:10.

“Dosage form” denotes a drug delivery system for administering a therapeutically effective amount of at least one proton pump inhibitor to a patient in need of therapy. The dosage form may be administered once-daily for treating gastrointestinal disorders.

In one embodiment, the invention provides drug releasing beads that on dissolution or diffusion release the proton pump inhibitor over 24 hours. The beads comprise a central composition or core comprising a proton pump inhibitor and pharmaceutically acceptable composition forming ingredients including an optional lubricant, antioxidant, and buffer. The beads are preparations with a general diameter of 1 mm to 2 mm. The beads comprise doses, as described herein, of proton pump inhibitor. The beads in one embodiment are formed of noncrossed-linked materials to enhance their discharge from the gastrointestinal tract. The beads are coated with a polymer that gives an extended release profile. The extended release beads can be manufactured into a tablet or inserted into a capsule. The beads, the tablet and/or the capsule may have an enteric coating as described above. The beads are made into matrix tablets by the direct compression of a plurality of beads coated with, for example, an acrylic resin and blended with excipients such as hydroxypropylmethyl-cellulose. The manufacture of beads is disclosed in Lu, Inter. J. of Pharm., 112:117-124 (1994); Pharm. Sci., by Remington, 14th Ed. pp. 1626-1628 (1970); Fincher, J. Pharm. Sci., 57:1825-1835 (1968); and U.S. Pat. No. 4,083,949. The manufacture of the tablet is described in Pharmaceutical Sciences, by Remington, 17th Ed., Chp. 90, pp. 1603-1625, (1985).

In another embodiment, the invention provides a proton pump inhibitor coated on a polymer substrate. The polymer can be an erodible, or a nonerodible polymer. The coated substrate is folded onto itself to provide a bilayer polymer drug dosage form. The drug dosage form may further comprise an enteric coating as described above. For example, the proton pump inhibitor is coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a rate to dispense a therapeutic dose of proton pump inhibitor over an extended period of time. Representative biodegradable polymers include, for example, poly(amides), poly(amino acids), poly(esters), poly(lactic acid), poly(glycolic acid), poly(carbohydrate), poly(orthoester), poly(orthocarbonate), poly(acetyl), poly(anhydrides), biodegradable poly(dehydropyrans), and poly(dioxinones). The polymers are known to the art in Controlled Release of Drugs, Rosoff, Chp. 2, pp. 53-95 (1989); and in U.S. Pat. Nos. 3,811,444; 3,962,414; 4,066,747, 4,070,347; 4,079,038; and 4,093,709.

In another embodiment, the invention provides a dosage form comprising a polymer that releases the proton pump inhibitor by diffusion through a polymer, or by flux through pores, or by rupture of a polymer matrix. The drug delivery polymeric dosage form comprises a proton pump inhibitor homogenously contained in or on a polymer. The dosage form comprises at least one exposed surface at the beginning of dose delivery. The nonexposed surface, when present, is coated with a pharmaceutically acceptable material impermeable to the passage of the proton pump inhibitor. The dosage form may further be partially or fully coated with an enteric coating layer to ensure safe passage of the proton pump inhibitor through the acidic stomach. The dosage form can be manufactured by procedures known to the art. An example of providing a dosage form comprises blending a pharmaceutically acceptable carrier (e.g., polyethylene glycol) with a known dose of proton pump inhibitor at an elevated temperature (e.g., 37° C.) and adding it to a silastic medical grade elastomer with a cross-linking agent (e.g., octanoate) followed by casting in a mold. The step is repeated for each optional successive layer. The system is allowed to set (e.g., for 1 hour) to provide the dosage form. Thereafter, an enteric coating may be applied. Representative polymers for manufacturing the dosage form include, for example, olefin and vinyl polymers, addition polymers, condensation polymers, carbohydrate polymers, and silicon polymers as represented by poly(ethylene), poly(propylene), poly(vinyl acetate), poly(methyl acrylate), poly(isobutyl methacrylate), poly(alginate), poly(amide), and poly(silicone). The polymers and manufacturing procedures are known in Polymers, by Coleman et al., Vol. 31, pp. 1187-1231 (1990); Drug Carrier Systems, by Roerdink et al., Vol. 9, pp. 57-109 (1989); Adv. Drug Delivery Rev., by Leong et al., Vol. 1, pp. 199-233 (1987); Handbook of Common Polymers, Compiled by Roff et al., (1971), published by CRC Press; and U.S. Pat. No. 3,992,518.

The invention provides a dosage form comprising a matrix comprising a plurality of tiny pills. The tiny pills provide a number of individual doses for providing various timed doses for achieving an extended release proton pump inhibitor delivery profile. The matrix comprises at least one hydrophilic polymer (e.g., a polysaccharide, agar, agarose, natural gum, alkali alginate including sodium alginate, carrageenan, fucoidan, furcellaran, laminaran, hypnea, gum arabic, gum ghatti, gum karaya, grum tragacanth, locust bean gum, pectin, amylopectin, gelatin, a hydrophilic colloid, and the like). The hydrophilic matrix comprises a plurality of 4 to 50 tiny pills. The tiny pills comprise a release rate controlling wall of 0.001 up to 10 mm thickness to provide for the timed release of proton pump inhibitor. Representative wall-forming materials include a triglyceryl ester (e.g., glyceryl tristearate, glyceryl monostearate, glyceryl dipalimitate, glyceryl laureate, glyceryl didecenoate, glyceryl tridenoate, and the like). Other wall forming materials comprise polyvinyl acetate phthalate, methylcellulose phthalate, and microporous vinyl olefins. Procedures for manufacturing tiny pills are disclosed in U.S. Pat. Nos. 4,434,153; 4,721,613; 4,853,229; 2,996,431; 3,139,383, and 4,752,470. The tiny pills may further comprise a full or partial enteric coating. Alternatively, the tiny pills may be placed in a capsule that comprises an enteric coating.

The invention provides a dosage form comprising a semipermeable wall that surrounds a therapeutic composition comprising at least one proton pump inhibitor. The semipermeable wall may be completely or partially surrounded by an enteric coating. In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over an extended period of time to provide an extended release of the proton pump inhibitor. In another embodiment, an osmotic dosage form comprises a wall surrounding a compartment, the wall comprising a semipermeable polymeric composition permeable to the passage of fluid and substantially impermeable to the passage of the proton pump inhibitor present in the compartment; a proton pump inhibitor drug layer composition in the compartment comprising the proton pump inhibitor; a hydrogel push layer composition in the compartment comprising an osmotic formulation for imbibing and absorbing fluid for expanding in size for pushing the proton pump inhibitor composition layer from the dosage form; and at least one passageway in the wall for releasing the proton pump inhibitor. The method delivers the proton pump inhibitor by imbibing fluid through the semipermeable wall at a fluid imbibing rate determined by the permeability of the semipermeable wall and the osmotic pressure across the semipermeable wall causing the push layer to expand; and thereby deliver the proton pump inhibitor from the dosage form through the exit passageway to a patient over an extended period of time.

The osmotic dosage forms in one manufacture comprise a therapeutic composition comprising at least one proton pump inhibitor, a pharmaceutically acceptable salt thereof and/or a stereoisomer thereof, and from about 1 mg to about 500 mg, of from about 10 mg to about 350 mg of a pharmaceutically acceptable hydrogel, such as a polyalkylene oxide of about 75,000 to about 750,000 weight-average molecular weight. Representative of polyalkylene oxides are polyethylene oxide of about 100,000 weight-average molecular weight, polyethylene oxide of about 200,000 weight-average molecular weight, polyethylene oxide of about 300,000 weight-average molecular weight, polyethylene oxide of about 600,000 weight-average molecular weight, and polypropylene oxide of about 100,000 weight average molecular weight. The therapeutic composition may also comprise 0 mg to about 100 mg, or from about 1 mg to about 50 mg of a hydroxypropylalkylcellulose of about 9,000 to about 150,000 average-number molecular weight selected from the group consisting of hydroxypropylmethylcellulose, hydroxypropylethylcellulose, hydroxypropylbutyl-cellulose, and hydroxypropylpentylcellulose, 0 to about 20 mg of a hydroxyalkyl-cellulose, such as hydroxypropylcellulose; 0 mg to about 100 mg, or from about 1 mg to about 50 mg, of an osmotic solute selected from the osmotically effective compounds such as sodium chloride, potassium chloride, potassium acid phosphate, tartaric acid, citric acid, raffinose, magnesium sulfate, magnesium chloride, urea, inositol, sucrose, glucose, sorbitol, and mixtures of two or more thereof; and 0 mg to about 8 mg, or from about 0.01 mg to about 5 mg of a lubricant, such as calcium stearate, zinc stearate, magnesium stearate, magnesium oleate, calcium palmitate, sodium suberate, potassium laureate, salts of fatty acids, salts of alicyclic acids, salts of aromatic acids, stearic acid, oleic acid, palmitic acid, and a mixture of salt of fatty, alicyclic or aromatic acid and a fatty, alicyclic or aromatic acid.

The invention provides for the composition to be surrounded by a wall comprising a semipermeable composition with an exit for delivering the composition to a human patient in need of proton pump inhibitor therapy. The invention provides, in an additional embodiment, the composition comprising at least one proton pump inhibitor as a therapeutic layer in layered, contacting arrangement with a hydrogel expansion composition manufactured as a layer that supports the therapeutic composition to yield a bilayered matrix. The composition may further comprise an enteric coating. The hydrogel layer composition may comprise about 1 mg to about 500 mg, or from about 10 mg to about 350 mg, of a hydrogel (e.g., a polyalkylene oxide of about 1,000,000 to about 8,000,000 which are selected from the group consisting of polyethylene oxide of about 1,000,000 weight-average molecular weight, a polyethylene oxide of about 2,000,000 molecular weight, a polyethylene oxide of about 4,000,000 molecular weight, a polyethylene oxide of about 5,000,000 molecular weight, a polyethylene oxide of about 7,000,000 molecular weight, a polypropylene oxide of the about 1,000,000 to about 8,000,000 weight-average molecular weight); or about 10 mg to about 250 mg of an alkali carboxymethylcellulose of about 10,000 to about 6,000,000 weight-average molecular weight (e.g., sodium carboxymethyl-cellulose or potassium carboxymethylcellulose).

The hydrogel expansion layer may further comprise 0.0 mg to about 350 mg, or from about 0.1 mg to about 250 mg, of a hydroxyalkylcellulose of about 7,500 to about 4,500,000 weight-average molecular weight (e.g., hydroxymethylcellulose, hydroxyethylcellulose, hydroxypropylcellulose, hydroxybutylcellulose, hydroxypentyl-cellulose); 0 mg to about 100 mg, or from about 1 mg to about 50 mg, of an osmagent (e.g., sodium chloride, potassium chloride, potassium acid phosphate, tartaric acid, citric acid, raffinose, magnesium sulfate, magnesium chloride, urea, inositol, sucrose, glucose, sorbitol); 0 to about 5 mg of a colorant, such as ferric oxide; 0 mg to about 50 mg, or from about 0.1 mg to about 30 mg, of a hydroxypropylalkylcellulose of 9,000 to 225,000 average-number molecular weight (e.g., hydroxypropylethylcellulose, hydroxypropypentylcellulose, hydroxypropylmethylcellulose, hydropropylbutylcellulose); 0 to about 1.5 mg of an antioxidant (e.g., ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, dibutylphenol, vitamin E, lecithin, ethanolamine); and 0 mg to about 10 mg of a lubricant (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

The invention provides for the composition, the therapeutic bilayer comprising the proton pump inhibitor layer, and the hydrogel layer to be administered as the composition or the bilayer per se; that is, as the composition or the bilayer together for treating gastrointestinal disorders. The invention provides additionally for the composition and for the compositional bilayer to be surrounded by a wall comprising a semipermeable composition with an exit for delivering the therapeutic composition to a patient in need of proton pump inhibitor therapy. The invention also provides for a subcoat to surround the therapeutic composition or to surround the bilayer, which subcoat in either embodiment is surrounded by a outer semipermeable wall. The composition may further comprise an enteric coating as described above.

The invention provides a dosage form comprising a wall, which wall surrounds an internal lumen or compartment. The wall comprises a semipermeable composition that is permeable to the passage of fluid and impermeable to the passage of proton pump inhibitor. The dosage form may further comprise an enteric coating. The wall is nontoxic and it comprises at least one polymer (e.g., cellulose acylate, cellulose diacylate, cellulose triacylate, cellulose acetate, cellulose diacetate, cellulose triacetate and the like). The wall comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether selected from the group consisting of hydroxypropylcellulose or hydroxypropylalkycellulose (e.g., hydroxypropylmethylcellulose). The total weight percent of all components comprising the wall is equal to 100 wt %. The internal compartment comprises the therapeutic proton pump inhibitor composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of proton pump inhibitor to a patient over an extended period of time. The dosage form comprises a passageway in the wall that connects the exterior of the dosage form with the internal compartment. The osmotic dosage form provided by the invention delivers proton pump inhibitor at an extended release rate. The dosage form may further comprise an enteric coating.

The expression “passageway” as used herein comprises means and methods suitable for the metered release of the proton pump inhibitor from the compartment of the dosage form. The exit means comprises at least one passageway, including orifice, bore, aperture, pore, porous element, hollow fiber, capillary tube, channel, porous overlay, or porous element that provides for the osmotic controlled release of the proton pump inhibitor. The passageway includes a material that erodes or is leached from the wall in a fluid environment of use to produce at least one controlled-release dimensioned passageway. The passageway may comprise enteric polymers as described above. Representative materials suitable for forming a passageway, or a multiplicity of passageways comprise a leachable poly(glycolic) acid or poly(lactic) acid polymer in the wall, a gelatinous filament, poly(vinyl alcohol), leachable polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of proton pump inhibitor from the dosage form. The dosage form can be constructed with one or more passageways in spaced apart relationship on a single surface or on more than one surface of the wall. The expression “fluid environment” denotes an aqueous or biological fluid as in a human patient. Passageways and equipment for forming passageways are disclosed in U.S. Pat. Nos. 3,845,770; 3,916,899; 4,063,064; 4,088,864 and 4,816,263. Passageways formed by leaching are disclosed in U.S. Pat. Nos. 4,200,098 and 4,285,987.

The wall of dosage forms can be formed by using an air suspension procedure. This procedure consists of suspending and tumbling the composition or the layers in a current of air and wall-forming composition until a wall is applied to the proton pump inhibitor forming compartment. The air suspension procedure is well suited for independently forming the wall. The air suspension procedure is described in U.S. Pat. No. 2,799,241; J. Am. Pharm. Assoc., Vol. 48, pp. 451-454 (1959); and ibid, Vol. 49, pp. 82-84 (1960). The wall can be formed with a wall-forming composition in a Wurster® air suspension coater using an organic solvent, such as acetone-water cosolvent 90:10 (wt:wt) with 2.5 wt % to 7 wt % polymer solids. An Aeromatic® air suspension coater using, for example, a methylene dichloride-methanol cosolvent comprising 87:13 (v:v) can be used for applying the wall. Other wall-forming techniques, such as pan coating system, wall forming compositions deposited by successive spraying of the composition or the bilayered arrangement, accompanied by tumbling in a rotating pan can be used for the present purpose. A larger volume of cosolvent can be used to reduce the concentration of polymer solids to produce a thinner wall. Finally, the wall of the coated compartments are laser or mechanically drilled, and then dried in a forced air or humidity oven for 1 to 3 days or longer to free the solvent. Generally, the walls formed by these techniques have a thickness of 2 to 20 mils (0.051 to 0.510 mm) with a preferred thickness of 2 to 6 mils (0.051 to 0.150 mm).

The dosage forms of the invention are manufactured by standard manufacturing techniques. For example, in one manufacture the proton pump inhibitor and other ingredients comprising a therapeutic composition or comprising the proton pump inhibitor composition that faces the exit means are blended, or they are blended then pressed into a composition. The proton pump inhibitor and other ingredients can be blended with a solvent and then formed into a solid or semisolid formed by conventional manufacturing methods such as ball-milling, calendaring, stirring, or roll-milling and then pressed into a selected shape. The composition possesses dimensions that correspond to the internal dimensions of the area it occupies in the dosage form. In the manufacture of bilayered compositions dosage form, the bilayers posses dimensions corresponding to the internal lumen of the dosage form. First, the hydrogel expansion layer is placed in contact with the proton pump inhibitor layer. The layering of the proton pump inhibitor layer and the hydrogel layer can be fabricated by conventional press-layering techniques. Finally, the two-layer compartment forming members are surrounded and coated with an outer wall. A passageway is drilled by laser or mechanically drilled through the wall, or the wall is provided with a pore-former to contact the proton pump inhibitor layer, with the dosage form optically oriented automatically by the equipment for laser forming the passageway on the preselected drug surface.

In another manufacture, the dosage forms are manufactured by the wet granulation technique. In the wet granulation technique the proton pump inhibitor and the ingredients comprising the drug composition are blended using an organic or inorganic solvent, such as isopropyl alcohol-methylene dichloride 80:20 (v:v) as the granulation fluid. Other granulating fluid, such as water, isopropyl alcohol, or denatured alcohol 100% can be used for this to purpose. The ingredients forming the drug composition are individually passed through a 40 mesh screen and then thoroughly blended in a mixer. Next, other ingredients comprising the drug composition are dissolved in a portion of the granulation fluid, such as the cosolvent described above. Then, the latter prepared wet blend is slowly added to the proton pump inhibitor blend with continual mixing in the blender. The granulating fluid is added until a wet blend mass is produced, which wet mass is then forced through a 20 mesh screen onto oven trays. The blend is dried for 18 to 24 hours at 25° C. to 40° C. The dry granules are then screened with a 16 mesh screen. Next, a lubricant is passed through an 60 mesh screen and added to the dry screened granule blend. The granulation is put into milling jars and mixed on a jar mill for 2 to 10 minutes. The first and second layer compositions are pressed into a layered tablet, for example, in a Manesty® layer press.

Another manufacturing process that can be used for providing a proton pump inhibitor and hydrogel composition comprises blending their powdered ingredients in a fluid bed granulator. After the powdered ingredients are dry blended in the granulator, a granulating fluid, for example, poly(vinylpyrrolidone) in a solvent, such as in water, is sprayed onto the respective powders. The coated powders are then dried in a granulator. This process coats the ingredients present therein while spraying the granulating fluid. After the granules are dried, a lubricant, such as stearic acid or magnesium stearate, is blended as above into the mixture. The granules are then pressed in the manner described above. In another embodiment, when the fluid bed granulating process is used to manufacture the hydrogel layer, the antioxidant present in the polyalkylene oxide can be removed during the processing step. If antioxidant is desired it can be added to the hydrogel formulation, and this can be accomplished during the fluid bed granulation process.

The dosage forms of this invention are manufactured in another embodiment by mixing the proton pump inhibitor with composition-forming ingredients and pressing the composition into a layer possessing dimensions that correspond to the internal dimensions of the compartment space adjacent to a passageway. In another embodiment, the proton pump inhibitor and other drug composition forming ingredients and a solvent are mixed into a solid, or semi-solid, by conventional methods such as ball-milling, calendaring, stirring or roll-milling, and then pressed into a preselected, layer-forming shape.

In the manufactures as presented above, the manufacture comprising a composition or comprising a layer of a composition comprising a hydrogel osmopolymer and an optional osmagent are placed in contact with the layer comprising the proton pump inhibitor, and the two layers comprising the layers are surrounded with a semipermeable wall. The layering of the first proton pump inhibitor composition and the second hydrogel osmopolymer and optional osmagent composition can be accomplished using a conventional two-layer tablet press technique. The wall can be applied by molding, spraying or dipping the pressed shapes into wall-forming materials. Another technique that can be used for applying the wall is the air suspension coating procedure. This procedure consists in suspending and tumbling the two layers in a current of air until the wall forming composition surrounds the layers. Manufacturing procedures are described in Modern Plastics Encyclopedia, Vol. 46, pp. 62-70 (1969); and in Pharmaceutical Sciences, by Remington, 14.sup.th Ed., pp. 1626-1680 (1970), published by Mack Publishing Co., Easton, Pa. The dosage form can be manufactured by following the teaching in U.S. Pat. Nos. 4,327,725; 4,612,008; 4,783,337; 4,863,456; and 4,902,514.

Exemplary solvents suitable for manufacturing the wall, the composition layers and the dosage form include inert inorganic and organic solvents that do not adversely harm the materials, the wall, the layer, the composition and the drug wall. The solvents broadly include members selected from the group consisting of aqueous solvents, alcohols, ketones, esters, ethers, aliphatic hydrocarbons, halogenated solvents, cycloaliphatics, aromatics, heterocyclic solvents, and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethylacetate, methylene dichloride, ethylene dichloride, propylene dichloride, carbon chloroform, nitroethane, nitropropane, tetrachloroethane, ethyl ether, isopropyl ether, cyclohexane, cyclo-octane, toluene, naphtha, 1,4-dioxane, tetrahydrofuran, diglyme, aqueous and nonaqueous mixtures thereof, such as acetone and water, acetone and methanol, acetone and ethyl alcohol, methylene dichloride and methanol, and ethylene dichloride and methanol.

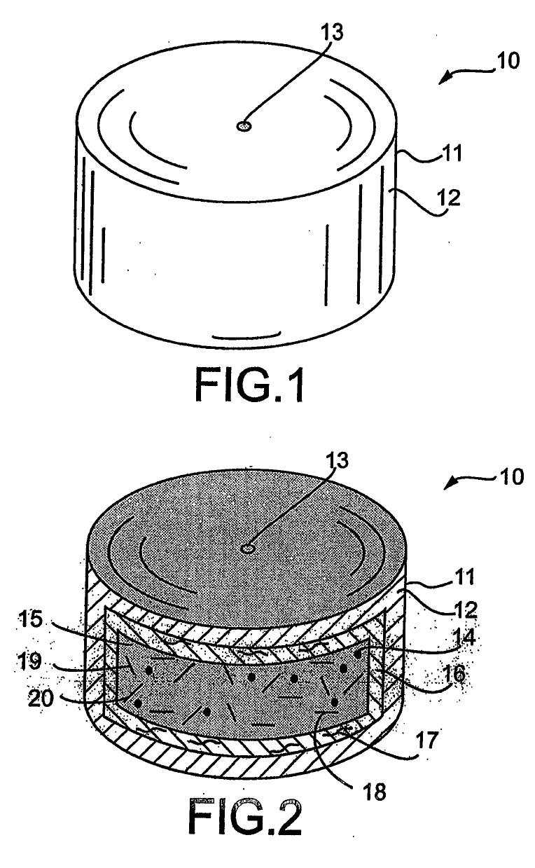

In another embodiment, the invention is represented by reference to FIGS. 1-3. The dosage forms exemplified in FIGS. 1-3 may further comprise an enteric coating or enteric polymer as shown, for example in FIGS. 37-40. The dosage form comprises a therapeutic composition of at least one proton pump inhibitor surrounded by a first coat and a second coat, or a dosage form comprising a therapeutic composition of at least one proton pump inhibitor and a push composition with both compositions being surrounded by a first coat and a second coat. After entering into the small intestine, the dosage form generates osmotic energy that causes the composition comprising the proton pump inhibitor to be administered through an exit port for an extended period of time.

In FIG. 1, a dosage form 10 comprises a body member 11 that comprises an exterior or second coat 12. The exterior or second coat 12 surrounds an interior or first coat and a compartment, not seen in FIG. 1. Dosage form 10 comprises at least one exit 13 that connects the exterior environment, such as the gastrointestinal tract of a human patient, with the interior of the dosage form.

In FIG. 2, dosage form 10 possesses extended release delivery kinetics. Dosage form 10 controls the extended delivery of proton pump inhibitor 14, represented by dots 14, from an internal space or compartment 15. Dosage form 10 delivers proton pump inhibitor 14 at a measured rate per unit time over an extended time.

Dosage form 10, as seen in FIGS. 1 to 3, is useful for establishing therapeutic drug levels in the blood, including the plasma, for therapy. Dosage form 10, as seen in the accompanying figures, embraces the shape of a dosage tablet, and it can embrace the shape of other orally administrable formulations. The extended release dosage form provides extended-continuous delivery greater than conventional, noncontrolled tablets, or noncontrolled-nonsustained release tablets and/or capsules that exhibit a dose-dumping of proton pump inhibitor.

Dosage form 10 of FIG. 2, comprises exterior, or second coat 12 that surrounds compartment 15. Second coat 12 comprises totally, or in at least a part, a semipermeable composition. The semipermeable composition is permeable to the passage of an aqueous or an aqueous-biological fluid, and second coat 12 is impermeable to the passage of proton pump inhibitor 14. Second coat 12 is nontoxic, and maintains its physical and chemical integrity during the dispensing time of proton pump inhibitor 14. The phrase, maintains its physical and chemical integrity means coat 12 does not lose its structure, and it does not undergo a chemical change during the dispensing of proton pump inhibitor 14.

Coat 12 comprises a composition that does not adversely affect the patient or components of the dosage form. Compositions for forming coat 12 are, in one embodiment, comprised of a cellulose ester polymer, a cellulose ether polymer, a cellulose ester-ether polymer, or a mixture of two or more thereof. These cellulosic polymers have a degree of substitution, DS, on the anhydroglucose unit, from greater than 0 up to 3 inclusive. By “degree of substitution” is meant the average number of hydroxyl groups originally present on the anhydroglucose unit comprising the cellulose polymer that are replaced by a substituting group. Representative coat 12 polymers include, for example, cellulose acylate, cellulose diacylate, cellulose triacylate, cellulose acetate, cellulose diacetate, cellulose triacetate, mono-, di- and tricellulose alkanylates, mono-, and di- and tricellulose alkinylates. Exemplary polymers include cellulose acetate having a DS of up to 1 and an acetyl content of up to 31%; cellulose acetate having a DS of 1 to 2 and any acetyl content of 21 to 35%; cellulose acetate having a DS of 2 to 3 and an acetyl content of 35 to 44.8%; and the like. More specific cellulosic polymers comprise cellulose propionate having a DS of 1.8, a propyl content of 39.2 to 45% and a hydroxyl content of 2.8 to 5.4; cellulose acetate butyrate having a DS of 1.8, an acetyl content of 13 to 15% and a butyryl content of 34 to 39%; cellulose acetate butyrate having a acetyl content of 2 to 29%, a butyryl content of 17% to 53% and a hydroxyl content of 0.5 to 4.7; cellulose triacylates having a DS of 2.9 to 3, such as cellulose trivalerate, cellulose trilaurate, cellulose tripalmitate, cellulose trisuccinate and cellulose trioctanoate; celluloses diacylate having a DS of 2.2 to 2.6, such as cellulose disuccinate, cellulose dipalminate, cellulose dioctanoate, cellulose dipentanoate, co-esters of cellulose, such as cellulose acetate butyrate, and cellulose acetate propionate.

Other semipermeable polymers include one or more of acetaldehyde dimethylcellulose acetate; cellulose acetate ethylcarbamate; cellulose acetate methylcarbamate; cellulose diacetate propylcarbamate; cellulose acetate diethylaminoacetate; semipermeable polyamide; semipermeable polyurethane; semipermeable sulfonated polystyrene; semipermeable crosslinked selective polymer formed by the coprecipitation of a polyanion and polycation, as disclosed in U.S. Pat. Nos. 3,173,876; 3,276,586; 3,541,005; 3,541,006 and 3,546,876; semipermeable polymers as disclosed in U.S. Pat. No. 3,133,132; semipermeable, lightly crosslinked polystyrenes; semipermeable crosslinked poly(sodium styrene sulfonate); semipermeable cross-linked poly(vinylbenzyltrimethyl ammonium chloride); and semipermeable polymers possessing a fluid permeability in the range of 2.5×10⁻⁸ to 5×10⁻²(cm²/hr·atm), expressed per atmosphere of hydrostatic or osmotic pressure difference across the semipermeable wall. The polymers are known to the polymer art in U.S. Pat. Nos. 3,845,770; 3,916,899 and 4,160,020; and in Handbook of Common Polymers, by Scott and Roff, 1971, CRC Press, Cleveland, Ohio. Second coat 12, in a present manufacture can be coated from a single solvent system, such as acetone.

Dosage form 10 comprises an interior or a first coat 16. The first coat 16 faces compartment 15, and second coat 12. Second coat 12 comprises a surface that faces the environment of use. First coat 16 comprises ethylcellulose, one hundred weight percent, (100 wt %), or in another manufacture a composition comprising a blend of 50 to 99 wt % ethylcellulose and 1 to 50 wt % hydroxypropylcellulose with the total weight of the compositional blend equal to 100 wt %. The first coat and the second coat are coated in a laminated arrangement free of heat and nonannealed to preserve the integrity and the properties of each coat. The ethylcellulose used for the first coat is nontoxic, insoluble in water, insoluble in intestinal fluid, and soluble in ethyl alcohol, and in a solvent system comprising ethyl alcohol and water. The ethylcellulose preferably comprises about 20 to about 60 weight percent ethoxy content, a viscosity of about 4 to about 200 centipose or higher, and about 5,000 to about 1,250,000 weight-average molecular weight. The hydroxypropylcellulose homogenously blended with the ethylcellulose is identified by a wave 17 in first coat 16. The hydroxypropylcellulose 17 comprises about 7,500 to about 1,500,000 weight average molecular weight, and is soluble in water below 40° C. and in ethyl alcohol.

In FIG. 2, internal compartment 15 comprises a single homogenous composition. The compartment 15 comprises at least one proton pump inhibitor 14, represented by dots.

Dosage form 10, in compartment 15 comprises a pharmaceutically acceptable hydrogel polymer 18, represented by level dashes. Representative polymer hydrogels include a maltodextrin polymer of the formula (C₆H₁₂O₅)_{λ}.H₂O, wherein λ is about 3 to about 7,500, and the maltodextrin polymer comprises a 500 to 1,250,000 number-average molecular weight; a poly(alkylene oxide) represented by a poly(ethylene oxide) and a poly(propylene oxide) having a 50,000 to 750,000 weight-average molecular weight, and more specifically represented by a poly(ethylene oxide) of at least one of 100,000, 200,000, 300,000, or 400,000 weight-average molecular weights; an alkali carboxyalkylcellulose, wherein the alkali is sodium, or potassium, or calcium, the alkyl is methyl, ethyl, propyl, or butyl of 10,000 to 1,000,000 weight-average molecular weight; and a copolymer of ethylene-acrylic acid, including methacrylic and ethacrylic acid of 10,000 to 500,000 number-average molecular weight. The therapeutic composition comprises about 5 to about 400 mg of a polymer hydrogel. The hydrogel polymer exhibits an osmotic pressure gradient across bilayer first coat and second coat thereby imbibing fluid into compartment 15 to form a solution or a suspension comprising drug 14 that is hydrodynamically and osmotically delivered from dosage form 10.

Dosage form 10 comprises a binder 19 represented by left-slanted dashes 19. The binder imparts cohesive qualities to the composition. Representative of materials for this invention useful as binders comprise a member selected from the group consisting of starch, gelatin, molasses, a vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate, methylcellulose, hydroxypropylcellulose, hydroxypropylmethylcellulose, and mixtures of binders. The binders can be used as a solution, or in a dry form to prepare the therapeutic composition. The therapeutic composition comprises 0 to about 100 mg of a binder, or from about 0.01 to about 25 mg of the binder.

Dosage form 10 comprises a lubricant 20 represented by right-slanted dashes 20. The lubricant is used during manufacture of the composition to prevent sticking to die walls or punch faces, generally to lessen adhesion. The lubricants are, for example, sodium stearate, oleic acid, potassium oleate, caprylic acid, sodium stearyl fumarate, magnesium palmitate, calcium stearate, zinc stearate, magnesium stearate, magnesium oleate, calcium palmitate, sodium suberate, potassium laureate, stearic acid, salts of fatty acids, salts of alicyclic acids, salts of aromatic acids, oleic acid, palmitic acid, a mixture of a salt of a fatty, alicyclic or aromatic acid, and/or a mixture of magnesium stearate and stearic acid. The amount of lubricant in the therapeutic composition is about 0.01 to about 20 mg.

FIG. 3 depicts dosage form 10 in opened section illustrating internal compartment 15. Internal compartment comprises the therapeutic composition containing proton pump inhibitor 14, as described in detail in FIG. 2. The therapeutic composition of FIG. 2 is identified further in FIG. 3 as drug layer 21. Drug layer 21 comprises the ingredients described in FIG. 2 and the details previously disclosed are included in this description of FIG. 3. Drug layer 21 in FIG. 3 initially is in contact with push layer 22.

In FIG. 3, push layer 22 comprises about 10 mg to about 400 mg of an expandable osmopolymer 23 represented by “v”. The osmopolymer 23 in layer 22 possesses a higher molecular weight than the hydrogel polymer 18 in the proton pump inhibitor composition. The osmopolymer 23 comprises a polyalkylene oxide and/or a carboxyalkylcellulose. The polyalkylene oxide possesses a 1,000,000 to 10,000,000 weight-average molecular weight. Representative of polyalkylene oxide include polymethylene oxide, polyethylene oxide, polypropylene oxide, polyethylene oxide having a 1,000,000 molecular weight, polyethylene oxide possessing a 2,000,000 molecular weight, polyethylene oxide comprising a 3,000,000 to 5,000,000 molecular weight, polyethylene oxide comprising a 7,000,000 and 7,800,000 molecular weight, cross-linked polymethylene oxide possessing a 1,000,000 molecular weight, and/or polypropylene oxide of 1,200,000 molecular weight. Typical osmopolymer 22 carboxyalkylcellulose in the expandable layer comprises a 200,000 to 7,250,000 weight-average molecular weight. Representative carboxyalkycellulose are alkali carboxyalkylcellulose, sodium carboxymethyl-cellulose, calcium carboxymethylcellulose, potassium carboxymethyl-cellulose, sodium carboxyethylcellulose, lithium carboxyalkylhydroxy-alkylcellulose, sodium carboxyethyl-cellulose, carboxyalkylhydroxy-alkylcellulose, carboxymethylhydroxyethylcellulose, carboxyethylhydroxy-ethylcellulose and carboxymethylhydroxypropylcellulose. The osmopolymers used for the push-expandable layer exhibit an osmotic pressure gradient across semipermeable coat 12. The osmopolymers imbibe fluid into dosage form 10, thereby swelling, expanding as a hydrogel or osmogel whereby they push the proton pump inhibitor from the osmotic dosage form.

Push layer 22 comprises 0 to about 75 mg, or from about 0.5 to about 75 mg of an osmotically effective compound 24, represented by circles. The osmotically effective compounds are known also as osmagents and as osmotically effective solutes. They imbibe an environmental fluid, for example, from the intestinal tract, into dosage form 10 for contributing to the delivery kinetics of push layer 21. Exemplary osmotically active compounds include osmotic salts, such as sodium chloride, potassium chloride, magnesium sulfate, lithium phosphate, lithium chloride, sodium phosphate, potassium sulfate, sodium sulfate, potassium phosphate, osmotic carbohydrates; glucose, fructose and maltose, urea, tartaric acid, potassium acid phosphate, citric acid, and a mixture of sodium chloride and urea.

Push layer 22 comprises 0 to about 75 mg of a suspending agent hydroxypropylalkylcellulose, represented by clear triangles 25. The hydroxypropylalkylcellulose comprises an alkyl of 1 to 7 carbons, straight or branched, with the hydroxypropylalkylcellulose possessing a 9,000 to 450,000 number-average molecular weight. The hydroxypropylalkylcellulose is, for example, hydroxypropylmethylcellulose, hydroxypropylethylcellulose, hydroxypropylisopropylcellulose, hydroxypropylbutylcellulose and/or hydroxypropylpentylcellulose. Push layer 22 optionally comprises a hydroxyalkylcellulose, also represented by triangles 25. The hydroxyalkylcellulose viscosity-increasing agent is hydroxymethylcellulose, hydroxyethylcellulose, hydroxypropylcellulose and hydroxybutylcellulose comprising a 7,500 to 150,000 viscosity-average molecular weight. The amount of hydroxyalkylcellulose is 0 to about 40 mg.

Push layer 22 comprises 0 to about 5 mg of a nontoxic colorant or dye 26 identified by vertical wavy lines. The colorant 26 makes the dosage form more esthetic in appearance, and it serves to identify the dosage form during manufacture and during therapy. The colorants include Food and Drug Administrations Colorant (FD&C), such as FD&C No. 1 blue dye, FD&C No. 4 red dye, FD&C yellow No. 5, FD&C yellow No. 6, FD&C blue No. 2, FD&C green No. 3, FD&C cranberry red No. 40, red ferric oxide, yellow ferric oxide, black ferric oxide, titanium dioxide, carbon black, indigo, and OPADRY® comprising polymers, polysaccharides, cellulose, starch and dye commercially available from Colorcon, West Point, Pa.

A lubricant 27, identified by half circles, is formulated into push-expandable layer 22. Exemplary lubricants include sodium stearate, potassium stearate, magnesium stearate, stearic acid, calcium stearate, sodium oleate, calcium palmitate, sodium laurate, sodium ricinoleate and/or potassium linoleate. The amount of lubricant is 0.01 to about 10 mg.

An antioxidant 28, represented by slanted dashes, is present in push-expandable formulation 22 to inhibit the oxidation of ingredients comprising expandable formulation 22. Expandable formulation 22 comprises 0 to about 5 mg of an antioxidant. Representative antioxidants include ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

Dosage form 10, comprises another manufacture provided by the invention. Dosage form 10 comprises an overcoat not shown on the outer surface of the wall of dosage form 10. The overcoat, which may be located beneath an enteric coating layer, is a therapeutic composition comprising about 0.01 to about 75 mg of proton pump inhibitor and about 0.5 to about 275 mg of a pharmaceutically acceptable carrier (e.g., alkylcellulose, hydroxyalkylcellulose and hydroxypropylalkylcellulose). The overcoat can be methylcellulose, hydroxyethylcellulose, hydroxybutylcellulose, hydroxypropylcellulose, hydroxypropylmethylcellulose, hydroxypropylethylcellulose and hydroxypropylbutylcellulose. The overcoat is formulated with 0 to about 50 weight percent of a plasticizer, opacificer, colorant, and/or antitact agents. The overcoat provides therapy immediately as the overcoat dissolves or undergoes dissolution in the presence of intestinal fluid and concurrently therewith delivers the proton pump inhibitor into the intestinal tract for immediate therapy.

Dosage form 10, manufactured as an extended release dosage form comprises at least one passageway 13. The expression “passageway” is described herein.

The enteric coat, first coat and the second coat of the dosage form can be formed using the air suspension procedure, pan coating system, wet granulations techniques, and other standard manufacturing techniques, as described herein.

### EXAMPLE 1

The therapeutic dosage form provided by the invention will be prepared as follows: first, rabeprazole sodium, mannitol, and polyethylene oxide of 100,000 weight-average molecular weight will be dry blended for 10 minutes in a 200 ml beaker, with mixing for 10 minutes with a stainless steel spatula. Next, the dry blend drug composition will be blended with 200 mg of magnesium stearate and the blended ingredients thoroughly blended to produce a homogenous drug composition. Next, the dry blend drug composition will be compressed into a single layer tablet. Then, 150 mg of the drug composition will be compressed under a pressure head of two-tons into a 9/32 inch (7.14 mm) diameter standard round tablet to provide the composition comprising the drug and the polyethylene oxide.

Next, the tablets will be transferred to a tablet coating machine, where they will be spray coated first with a solution of ethylcellulose comprising a 158,000 weight-average molecular weight and hydroxypropylcellulose comprising a number-average molecular weight of 85,000 in a solvent comprising ethanol and water. The percent ratio of ethylcellulose to hydroxypropylcellulose will be 55 to 45, respectively. The coating solution will be sprayed around the tablets to apply the first coat to a thickness of 5 mils (0.127 mm). Next, the tablets will be coated with a 2 mil second coat comprising cellulose acetate comprising an acetyl content of 38.5% and a 40,000 weight-average molecular weight and polyethylene glycol of 400 molecular weight dissolved in acetone, to form the second coat. The present ratio of cellulose acetate to polyethylene glycol will be 70 to 30, respectively. The dual coated dosage forms will be air dried at 25° C. and a passageway will be drilled through the dual coats to connect the drug composition with the exterior of the dosage forms. Thereafter, an enteric coating may be applied to the dosage forms.

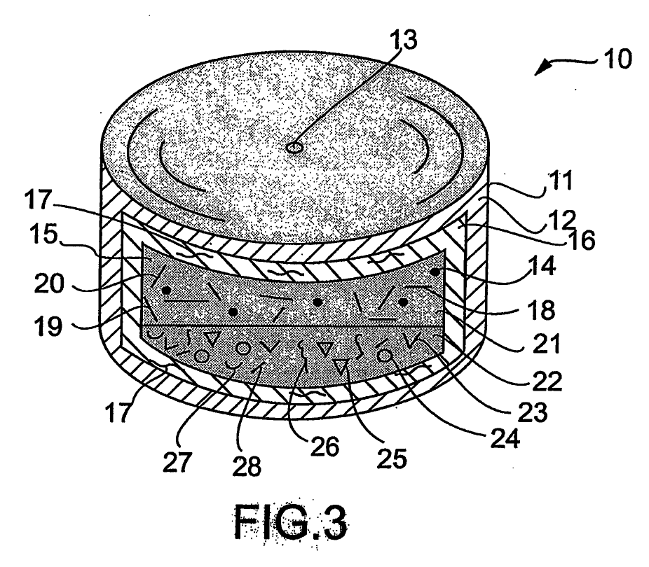

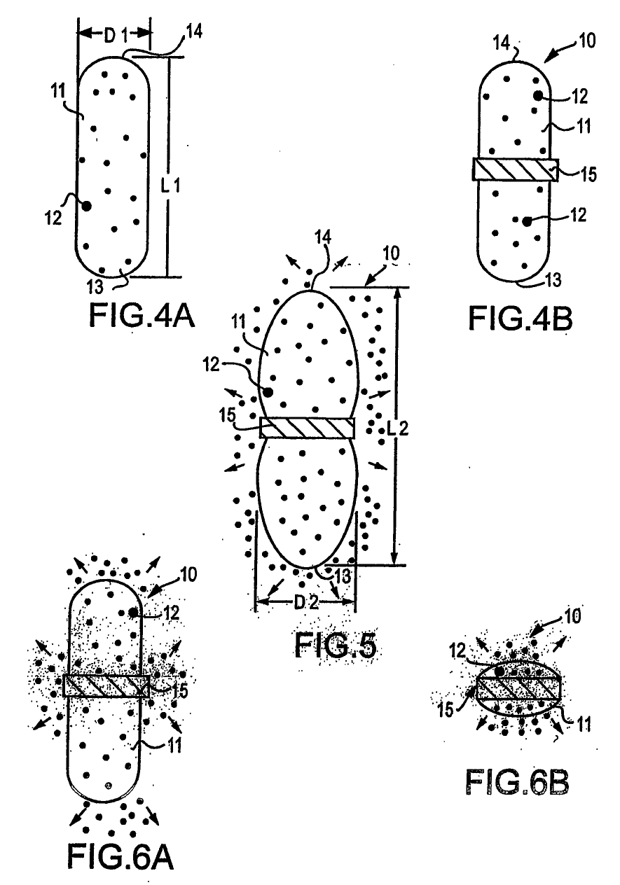

In another embodiment, the invention is represented by reference to FIGS. 4-10. The dosage forms exemplified in FIGS. 4-10 may further comprise an enteric coating or enteric polymer as shown, for example, in FIGS. 37-41.

The invention will be better understood with reference to the drawings and the description herein. FIG. 4 depicts one embodiment of the dosage form 10 according to the invention. The dosage form 10 comprises a polymer matrix 11 having at least one proton pump inhibitor 12 (illustrated by the multitude of dots) dissolved or dispersed therein. Polymer matrix 11 typically is formed of combination of a swellable polymer and a hydroattractant. The term “swellable” means, with respect to a polymer or a polymer matrix, that the polymer or polymer matrix is capable of imbibing fluid and expanding when in contact with fluid present in the environment of use. The polymer matrix can further comprise other pharmaceutically acceptable carriers that are used in conjunction with the proton pump inhibitor and/or with the polymers.

Exemplary swellable polymers are polyethylene oxide and cellulosic polymers (e.g., hydroxypropyl cellulose, hydroxypropyl methyl cellulose, hydroxyethyl cellulose, sodium carboxy methylcellulose, calcium carboxymethyl cellulose, methyl cellulose), and noncellulosic polymers (e.g., maltodextrin, polyvinyl alcohol, polyacrylic acids, alginates, gelatin, natural gums, including guar), lightly crosslinked versions of the cellulosic and noncellulosic polymers, starch graft copolymers and the like. The swellable polymers generally have number average molecular weights over 50,000 grams per mole, such as between 50,000 and 10,000,000 grams per mole and representative viscosities, e.g., for polyethylene oxide in the range of 12-20,000 cps (5% aq, 25° C., MW 100,000-900,000), 400-4000 cps (2% aq, 25° C., MW 1,000,000-2,000,000) and 1500-15,000 cps (1% aq, 25° C., MW 4,000,000-8,000,000) [Brookfield viscometer, rotational spindle]; for methylcellulose in the range of 1,500-18,000 cps (2% aq, 20° C., MW 62,000-134,000) [Ubbelohde tube viscometer]; for hydroxypropyl methylcellulose in the range of 4,000-100,000 cps (2% aq, 20° C., MW 88,000-242,000) [Ubbelohde tube viscometer]; for hydroxyethyl cellulose in the range of 75-400 cps (5% aq, 25° C., MW 90,000-200,000), 400-6500 cps (2% aq, 25° C., MW 300,000-720,000) and 1500-5,000 cps (1% aq, 25° C., MW 1,000,000-1,300,000) [Brookfield viscometer, rotational spindle]; for guar about 5100 cps (1%) [Brookfield viscometer, rotational spindle]; for poly(methyl vinyl ether/maleic anhydride) in the range of 15 to greater than 200 cps (5% aq., MW 20,000-80,000) [Brookfield viscometer, rotational spindle]; for polyvinyl alcohol in the range 27-65 cps (4% aq, 20° C. [Hoeppler falling ball method and 1100-1500 cps (10% aq, 25° C.) [Brookfield viscometer, rotational spindle; for sodium carboxymethyl cellulose in the range of 25-50 cps (2% aq, 25° C.) (MW 90,000) to about 2,500-6,000 cps (1% aq, 25° C.) (MW 700,000) [Brookfield viscometer, rotational spindle]; and for sodium polyacrylic acid 5000-75,000 (0.5% aq) (MW 750,000-4,000,000) [Brookfield viscometer, rotational spindle]. Polymers having molecular weights between 300,000 and 8,000,000 grams per mole are preferred, and those having molecular weights between about 5,000,000 to 8,000,000 grams per mole are especially preferred. Polyethylene oxide having a number average molecular weight between about 5,000,000 to 8,000,000 grams per mole is most especially preferred, e.g. Polyox 308. Also, especially preferred are methylcellulose type/grade A15C, A18M and hydroxypropyl methylcellulose type/grade K4M, K15M, 100 M and F4M (Dow Chemical Company); hydroxyethyl cellulose such as Natrosol® HEC; hydroxypropyl cellulose such as Klucel (Grades H, M, G, J, L, E-Aqualon Company); guar such as Supercol® Guar U (Aqualon Company); pectin such as GENU Pectin (Aqualon Company); carrageenan such as GENU Carrageenan (Aqualon Company); poly(methyl vinyl ether/maleic anhydride) such as Gantrez® AN Copolymer (AN-119, -139, -149, -169, -179, GAF Corporation); polyvinyl alcohol such as Elvanol® 71-30, Elvanol® 85-30, Elvanol® 50-42 and Elvanol® HV (DuPont); sodium carboxymethyl cellulose such as Aqualon cellulose gum grade 7H4; sodium polyacrylic acid such as Carpobol® resin grade 934PNF; and polyacrylic acid such as Carpobol® resin grade 934P.

Exemplary hydroattractants are water-insoluble polymers such as low substituted hydroxypropyl cellulose, microcrystalline cellulose (Avicel), cross-linked sodium or calcium carboxymethyl cellulose, cellulose fiber (Solka-Floc or Elcema), cross-linked polyvinyl pyrrolidone (Polyplasdone XL), cross-linked Amberlite resin, alginates (Satialgine), colloidal magnesium-aluminum silicate (Veegum), corn starch granules, rice starch granules, potato starch granules, wheat starch granules, sodium carboxymethyl starch (Expotab, Primojel), corn starch/acrylamide/sodium acrylate copolymer, acrylamide/sodium acrylate copolymer and the like. A particularly suitable hydroattractant is hydroxypropyl cellulose having a hydroxypropyl content of about 8 to about 15 weight percent, and preferably about 10 to about 13 weight percent, such as that supplied as Low Substituted Hydroxypropyl Cellulose grade 11 as manufactured by Shin-Etsu Chemical Company, Ltd., Tokyo, Japan.

Typically, the swellable polymer in the polymer matrix is present in from about 5% to about 90% by weight based on the total weight of the proton pump inhibitor dosage form, and the hydroattractant is present in from about 5% to about 70% by weight based on the total weight of the proton pump inhibitor dosage form. The particular percentages will be chosen to provide the desired retention time in the intestine(s) and the desired extended release profile of the proton pump inhibitor. However, it is preferred to have the polymer matrix contain from about 10 weight percent to about 50 weight percent (or from about 10 to about 40 weight percent) of the swellable polymer and from about 10 weight percent to about 60 weight percent (or from about 25 to about 35 weight percent) of the hydroattractant. In a preferred embodiment, the invention comprises a polymer composition having from about 10 weight percent to about 50 weight percent of a polyethylene oxide polymer and from about 10 weight percent to about 60 weight percent of a water-insoluble hydroxypropyl cellulose polymer. The polyethylene oxide polymer has a molecular weight of between about 100,000 and 10,000,000 grams per mole. The hydroxypropyl cellulose polymer preferably has a hydroxypropyl content of between about 8-15 weight percent, and most preferably between about 10-13 weight percent.

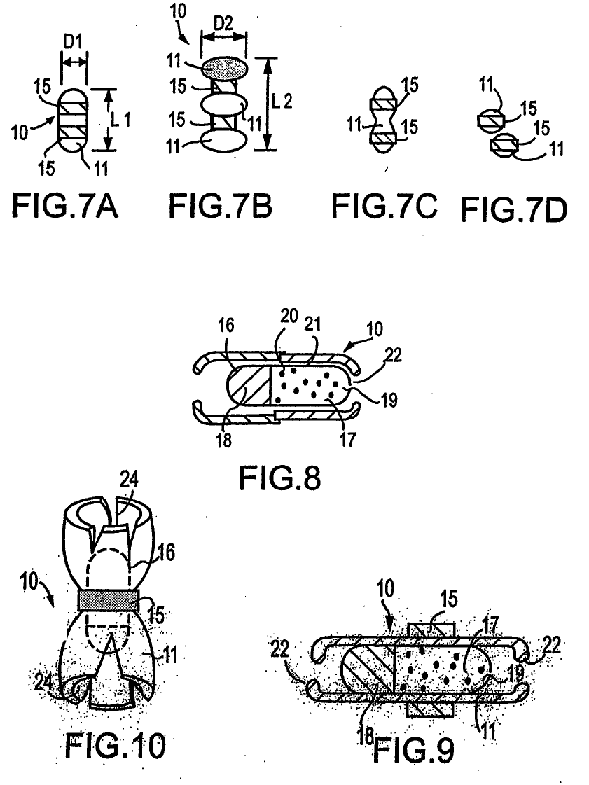

Dosage form 10 is conveniently cylindrically shaped with rounded ends 13 and 14 that facilitate administration of the dosage form in its non-swelled state. In FIG. 4A, the dosage form 10 is shown in preparation prior to application of the optional insoluble material or band 15 shown in FIG. 4B. The insoluble material exemplified as band 15, circumscribes a portion of the outer surface of the polymer matrix 11. While a single band is illustrated in FIG. 4, additional bands such as illustrated in FIG. 7 can optionally be used.

The band of insoluble material 15 is applied to the outer surface of the polymer matrix. The insoluble material imparts rigidity to the polymer matrix to manage intestinal retention time and further control the delivery profile of the proton pump inhibitor. Band 15 typically exhibits low water permeability and will prevent that portion of the polymer matrix which it surrounds from imbibing fluid, thus substantially limiting any swelling of polymer matrix 11 at that location. The number, size, and placement of the insoluble bands that are applied onto the surface of the proton pump inhibitor dosage form may be varied to adjust the proton pump inhibitor delivery profile and the retention time in the intestine(s). For example, bands 0.1 mm to about 12 mm in width, preferably between about 0.5 and about 8 mm, may be applied onto the surface of the proton pump inhibitor dosage form. Further, between about 1 and about 10 bands may be used, but generally between about 1 and about 3 are used. The bands may be placed close together (i.e., within about 0.5 mm of each other) or may be placed about 8 to about 12 mm apart.

With reference to FIGS. 7A-7D, dosage form 10 is formed with two bands 15, each circumscribing a portion of the surface of polymer matrix 11 in which the proton pump inhibitor (not shown) is dispersed. FIG. 7A illustrates dosage form 10 in its initial configuration before it has imbibed any fluid. Thereafter, dosage form 10 swells as shown in FIG. 7B in those segments of polymer matrix 11 that are not surrounded by bands 15. Because of the low fluid impermeability of bands 15, those portions of polymer matrix 11 surrounded by bands 15 do not appreciably imbibe fluid and the polymer in such segments of the polymer matrix does not swell to any significant extent. FIGS. 7C and 7D illustrate sequential states of dosage form 10 after it is substantially eroded by intestinal fluid. Eventually, dosage form 10 will separate into two pieces and be expelled from the intestine(s).

An alternate embodiment of the invention is illustrated in FIGS. 8-10 wherein a separate proton pump inhibitor containing reservoir is utilized with the polymer matrix 11 of the invention. The polymer matrix 11 is formed in the shape of a tube or annulus, for example by extrusion of the polymer mixture after preparation as described below, and positioned around a proton pump inhibitor reservoir denoted generally as 16. Reservoir 16 will be adapted to deliver proton pump inhibitor to the environment of use over an extended period of time. In one form it may be an osmotic pump, such as that manufactured as the OROS® active agent dispensers. Various types of osmotic dispensers include elementary osmotic pumps, such as those described in U.S. Pat. No. 3,845,770, mini-osmotic pumps such as those described in U.S. Pat. Nos. 3,995,631, 4,034,756 and 4,111,202, and multi-chamber osmotic systems referred to as push-pull, push-melt and push-stick osmotic pumps, such as those described in U.S. Pat. Nos. 4,320,759, 4,327,725, 4,449,983, 4,765,989 and 4,940,465, all of which are incorporated herein by reference. In the multi-chamber osmotic systems, the proton pump inhibitor reservoir 16 is typically formed with a proton pump inhibitor compartment 17, containing proton pump inhibitor in the form of a solid, liquid or suspension, as the case may be, and a compartment 18 of a hydrophilic polymer that will imbibe fluid from the intestine(s), swell and force the proton pump inhibitor from opening 19. Such osmotic pumps are sold commercially and have been described in the patents noted above and other patent and scientific literature. Other proton pump inhibitor delivery systems could be used, but the osmotically-driven systems are preferred for their well controlled delivery.

Polymer matrix 11 will swell in the intestine(s) and facilitate retention of the proton pump inhibitor reservoir 16 in the intestine(s) during the time that the proton pump inhibitor is being delivered. With reference to FIG. 8, polymer matrix 11 may be prepared in two parts and joined about the active agent reservoir 16, such as with complementary male ridges 20 and female grooves 21. More simply, it may be prepared in one piece as a tube or annular ring that is fitted or molded about the proton pump inhibitor reservoir 16 as shown in FIG. 9. Additionally, band(s) 15 may be placed about the polymer matrix, limiting the swelling of the polymer matrix in the segment surrounded by the band. Preferably, the tube or ring is formed with split ends 24 as illustrated in FIG. 10, such that upon swelling of the polymer matrix, the ends flare outwardly and create an effective diameter larger than that created in the case where the tube ends are not split. Conveniently, the polymer matrix 11 is injection molded about the proton pump inhibitor reservoir, or alternatively, it may be formed as a tube into which the proton pump inhibitor reservoir is inserted. In certain circumstances, it may be beneficial to have the polymer matrix extend past the end of the proton pump inhibitor reservoir, in which case the tube ends may be crimped to assist in retaining the proton pump inhibitor reservoir within the tube.

If a snug fit is provided initially between the inner surface of the polymer tube or annular ring and the outside wall of the proton pump inhibitor reservoir, then the swelling of the polymer matrix normally will be sufficient to retain the tube or annular ring on the proton pump inhibitor reservoir without any additional means being required. Intestinal fluid will contact the proton pump inhibitor reservoir and the proton pump inhibitor will be dispensed from hole 19 in the reservoir out through holes 22 that are present in the end of the polymer tube 11. While a single hole in the tubular polymer matrix 11 is considered adequate, it is preferred to have a hole at each end of the polymer tube. In another embodiment, illustrated in FIG. 9, the polymer matrix 11 is constrained by a band of insoluble material 15, as has been described. Band 15 constrains the polymer matrix and assists in retaining the polymer on the proton pump inhibitor reservoir 16. Either alone or in combination with band 15, the proton pump inhibitor reservoir 16 provides a rigid segment of the dosage form of the invention that facilitates the dosage form being retained in the intestine(s) for an extended period of time. When the polymer matrix has eroded, band 15 will release from the dosage form and be expelled.

The insoluble material comprising band(s) 15 may be any material that is nontoxic, biologically inert, nonallergenic and nonirritating to body tissue, that exhibits little impermeability to liquids, and that maintains its physical and chemical integrity in the environment of use for at least a portion of the dispensing period. The low liquid permeability of the insoluble material serves to limit swelling of the polymer matrix in that section of the polymer matrix that is surrounded by the band.

Insoluble materials from which the bands may be prepared include, for example, polyethylene, polystyrene, ethylene-vinyl acetate copolymers, polycaprolactone and Hytrel® polyester elastomers (Du Pont). Additional banding materials include but are not limited to polysaccharides, cellulosics, cellulose acetate, cellulose acetate propionate, cellulose acetate butyrate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease® as supplied by Colorcon, West Point, Pa. or Aquacoat® as supplied by FMC Corporation, Philadelphia, Pa.), nitrocellulose, polylactic acid, poly-glycolic acid, polylactide glycolide copolymers, polycaprolactone, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit® supplied by RohmPharma, Weiterstadt, Germany), polypropylene, copolymers of propylene oxide and ethylene oxide, propylene oxide ethylene oxide block copolymers, ethylenevinyl alcohol copolymer, poly sulfone, ethylene vinylalcohol copolymer, polyxylylenes, polyamides, rubbers, such as styrenebutadiene, polyisobutylene and the like, natural and synthetic waxes, paraffin, carnauba wax, petroleum wax, white or yellow bees wax, castor wax, candelilla wax, rice bran wax, microcrystalline wax, stearyl alcohol, cetyl alcohol, bleached shellac, esterified shellac, chitin, chitosan, silicas, polyalkoxysilanes, polydimethyl siloxane, polyethylene glycol-silicone elastomers, crosslinked gelatin, zein, electromagnetic irradiation crosslinked acrylics, silicones, or polyesters, thermally crosslinked acrylics, silicones, or polyesters, butadiene-styrene rubber, glycerol ester of partially dimerized rosin, glycerol ester of partially hydrogenated wood rosin, glycerol ester of tall oil rosin, glycerol ester of wood rosin, pentaerythritol ester of partially hydrogenated wood rosin, pentaerythritol ester of wood rosin, natural or synthetic terpene resin and blends of the above.

The banding materials often are also formulated with plasticizers, and optionally with wetting agents, surfactants, opacifiers, colorants, flavorants, taste-masking agents, and the like. Examples of typical plasticizers are as follows: polyhydric alcohols, polyethylene glycol, glycerol, propylene glycol, acetate esters, glycerol triacetate, triethyl citrate, acetyl triethyl citrate, glycerides, acetylated monoglycerides, oils, mineral oil, castor oil and the like.

Referring again to FIG. 4A, the polymer matrix 11 in its non-swelled state has a length L1 and a maximum diameter D1 intermediate the ends 13 and 14. FIG. 5 shows dosage form 10 after having been delivered to the small intestine. The polymer matrix 11 on each side of the band 15 has swelled from imbibing fluid from the intestine and begun to erode, thereby releasing proton pump inhibitor 12. In contrast to the exposed segments of the swollen polymer matrix 11, band 15 and the portion of the polymer matrix beneath it have not swelled to such an extent. Accordingly, that segment of the polymer matrix surrounded by band 15 is maintained in a constrained and more compressed, non-swollen state than the unbanded portion of the matrix. Since band 15 does not take up an appreciable amount of fluid from the intestine and swell, band 15 retains its substantially rigid or semi-rigid form, and provides an element of rigidity to the dosage form as a whole.

FIGS. 6A and 6B show dosage form 10 after a length of time in the fluid environment of the intestine(s). Polymer matrix 11 has eroded at the exposed surface of the matrix, i.e., those portions of the matrix not covered by the insoluble material 15 to such an extent that the dosage form 10 is smaller than its initial swollen configuration. Erosion of the polymer matrix will continue to deliver proton pump inhibitor to the intestine until the polymer matrix has substantially eroded so that no significant amount of proton pump inhibitor remains or has eroded to such an extent that the remainder of the dosage form is expelled from the intestine(s). Band 15 will be expelled either alone if it has separated from the dosage form at some time near the end of the delivery period or as part of the remainder of the dosage form expelled. In some applications, it may be desirable to form band 15 with weakened portions so that band 15 splits and falls away from the polymer matrix after some predetermined time to permit a particular release pattern of proton pump inhibitor from the dosage form over the delivery period.

The polymer matrices useful in this invention can be prepared by standard methods from the materials previously described. Typically, for example, an appropriate quantity of proton pump inhibitor and the polymer ingredients are separately passed through a screen, such as a screen having a mesh of about 40 wires per inch, to reduce any larger sized materials, and dry mixed. Then, a pharmaceutically-acceptable liquid, having a sufficient vapor pressure to allow subsequent drying over a reasonable period of time, for example 24 hours, is added to the dry mixture and the damp mass is extruded through a mesh screen (e.g. 20 wires per inch) to further mix the materials. Examples of suitable liquids are water, methanol, ethanol, isopropanol, acetone, and the like. After the extrusion process, the mixture is allowed to dry, for example in air overnight at room temperature if the proton pump inhibitor does not require any special handling. After drying, the resulting material is granulated, for example by passing the dried material through a mesh screen (e.g., 20 wires per inch). The granules are combined with a suitable tableting lubricant which has been previously passed through a mesh screen (e.g., 60 wires per inch). The resulting material is tumbled to produce the finished granulation for the tableting process. Tablets are produced using well known methodologies associated with horizontal and vertical compression units using dies and punches of appropriate dimensions. Alternate granulation methods, for example, fluid bed granulation or direct compression granulation can be used as well and such method will be chosen by one skilled in the art depending on the particular nature of the materials being used and the convenience and preference of the fabricator.

In order to prepare a dosage form of the invention, the proton pump inhibitor is first prepared and formed into a matrix of the desired size and shape. The matrix in its initial prepared form is about the size and dimensions of a size “000” to size 5 hard gelatin capsule, which may have an enteric coating. The cross-sectional shape of the matrix may be circular or may be oval, triangular, square, hexagonal or other shapes that are easily handled. The ring or bands are then placed onto the surface of proton pump inhibitor formulation matrix or printed onto the surface using conventional banding or printing techniques, such as disclosed herein or in U.S. Pat. No. 5,534,263, which is incorporated herein by reference.

As described above, the proton pump inhibitor itself may be in liquid, solid or semisolid form. The proton pump inhibitor formulation may contain additional materials and may be designed in a multitude of ways to provide a specific proton pump inhibitor delivery profile. In one embodiment, the polymer matrix may contain a surfactant so that the dosage form is more readily susceptible to erosion in the intestine. In still another embodiment, the dosage form may include a solid surfactant and provide proton pump inhibitor delivery in a finely dispersed form. In yet a further embodiment, the dosage form may include coated microspheres of a proton pump inhibitor or microspheres of a proton pump inhibitor and an adjuvant. The proton pump inhibitor either alone or with adjuvant can be delivered simultaneously from the microspheres either by diffusion or by osmosis. Suitable materials useful as proton pump inhibitor carriers and excipients are known in the art and are disclosed in U.S. Pat. Nos. 4,595,583 and 4,874,388, for example.

The proton pump inhibitor dosage form of this invention will preferably have a relative absorption index of at least 0.5, more preferably at least 0.8, even more preferably at least 1.0 and most preferably at least 1.2. The specific amount of proton pump inhibitor to be included in the dosage form of the invention can easily be determined by routine dosage studies that compare the blood plasma active agent levels of subjects with conventional dosing and the dosage form of this invention. The dosage forms of this invention can conveniently release proton pump inhibitor in an extended release manner over a prolonged period of time. Typically, the proton pump inhibitor will be released from the dosage form at a rate that releases a therapeutically effective amount of proton pump inhibitor to the subject over a substantial portion of the period between administration of the dosage forms.

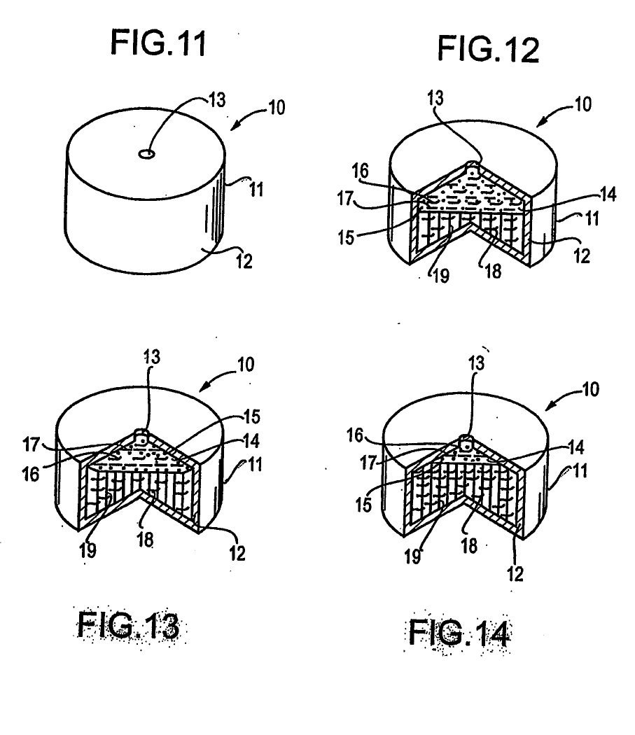

Another embodiment of the invention is represented by reference to FIGS. 11-15. The dosage forms exemplified in FIGS. 11-15 may further comprise an enteric coating or enteric polymer as shown, for example, in FIGS. 37-41.

In FIG. 11, dosage form 10 is seen comprising a body member 11 having a wall 12 and at least one passageway 13 for releasing a proton pump inhibitor from dosage form 10 to a fluid environment of use. The phrase “fluid environment of use” denotes the intestinal tract, comprising the intestine(s), and other fluid containing areas.

In FIG. 12, dosage form 10 of FIG. 11 is seen in opened section. In FIG. 12, dosage form 10 comprises a body 11, a wall 12 that surrounds and forms internal compartment 14, that communicates through a passageway 13 with the exterior of dosage form 10. Wall 12 comprises a semipermeable composition or at least in part a semipermeable composition. When wall 12 comprises at least in part a semipermeable composition the remainder of the wall is comprised of a non-semipermeable composition. Compartment 14 contains a first composition comprising a proton pump inhibitor 15, represented by dots, an osmopolymer 17, represented by horizontal dashes, that imbibes and/or absorbs fluid into compartment 14 and exhibits an osmotic pressure gradient across semipermeable wall 12 against an exterior fluid present in the environment of use, and an optional osmagent 16, represented by irregular lines, that is soluble in fluid imbibed into compartment 14 and exhibits an osmotic pressure gradient across semipermeable wall 12 against an external fluid and. Wall 12 comprises a semipermeable composition that is substantially permeable to the passage of the exterior fluid, and it is substantially impermeable to the passage of the proton pump inhibitor 15, osmagent 16 and osmopolymer 17. Semipermeable wall 12 is non-toxic and it maintains its physical and chemical integrity during the delivery life of the proton pump inhibitor 15 from dosage form 10.

Compartment 14 also houses a second composition that is distant from passageway 13 and in spaced relation with the first composition. The second composition contributes an expandable driving force that pushes and acts in cooperation with the first composition for delivering the maximum amount of proton pump inhibitor 15 from dosage form 10. The second composition comprises an osmopolymer 19, represented by vertical lines, that imbibes fluid into compartment 14 and exhibits an osmotic pressure gradient across semipermeable wall 12 against external fluid blended with, optionally, an osmagent 18, represented by wavy lines, that is soluble in fluid imbibed into compartment 14 and exhibits an osmotic pressure gradient across semipermeable wall 12 against an external fluid.

Osmopolymer 17 and osmopolymer 19 are hydrophilic water soluble or lightly cross-linked water soluble polymers, and they possess osmotic properties such as the ability to imbibe external fluid through the semipermeable wall, exhibit an osmotic pressure gradient across the semipermeable wall against the external fluid, and swell or expand in the presence of the fluid in the compartment. Osmopolymers 17 and 19 preferably are mixed with an optional osmagent 16 and an optional osmagent 18, respectively, for imbibing the optimal maximum volume of external fluid into compartment 14. This imbibed fluid is available to osmopolymers 17 and 19 to optimize the volumetric rate and for total expansion of osmopolymer 17 and 19. That is, osmopolymers 17 and 19 absorb fluid imbibed into compartment 14 by the osmotic imbition action of osmopolymers 17 and 19 supplemented by the osmotic imbition action of optional osmagents 16 and 18 for effecting the optimal maximum expansion of osmopolymers 17 and 19 from a rested to an enlarged, that is, an expanded state.

In operation, the delivery of proton pump inhibitor 15 from osmotic dosage form 10 is carried out, in one embodiment, by (1) imbibition of fluid by the first composition to form a fluidic composition in situ and delivery of the suspension through the passageway; and concurrently by (2) imbibition of fluid by the second composition causing the second composition to swell and cooperate with the first composition for driving the proton pump inhibitor formulation through at least one, or more than one, passageways.

The proton pump inhibitor composition can be delivered as a ribbon, which is a viscous or paste-like strip. According to the operation described, the dosage form may be considered as a cylinder, with the second composition expanding like the movement of a piston for aiding in delivering the proton pump inhibitor composition from the dosage form.

The volume rate delivered by the dosage form 10 Fₜ is composed of two sources: the water imbibition rate by the first composition F, and the water imbibition rate by the second composition Q wherein: Fₜ=F+Q (1)

Since the boundary between the first composition and the second composition hydrates very little during the functioning of the dosage form, there is insignificant water migration between the compositions. Thus, the water imbibition rate of the second composition, Q, equals the expansion of its volume: (dvₚ÷dt)=Q (2)

The total delivery rate from the osmotic dosage form is then,

(dm÷dt)=Fₜ·C═(F+Q)C (3)

wherein C is the concentration of proton pump inhibitor in the delivered slurry or solution. Conservation of the osmotic dosage form volume, V, and the surface area, A, gives equations (4) and (5): V=V_{d}+Vₚ (4); A=A_{d}+Aₚ (5); wherein V_{d} and Vₚ equal the volumes of the first composition and the second composition, respectively; and wherein A_{d} and Aₚ equal the surface area in contact with the wall by the first composition and the second composition, respectively. In operation, both Vₚ and Aₚ increase with time, while V_{d} and A_{d} decrease with time as the dosage form delivers the proton pump inhibitor.

The volume of the second composition that expands with time when fluid is imbibed into the compartment is given by equation (6): Vₚ=f(W_{H}÷Wₚ) (6), wherein W_{H} is the weight of fluid imbibed by the second composition, Wₚ is the weight of the second composition initially present in the dosage form, W_{H}/Wₚ is the ratio of fluid to initial solid of the second composition, and Vₚ=(1+(W_{H}÷Wₚ))(W_{H}+ρ) (7), wherein ρ is the density of the second composition corresponding to W_{H}/Wₚ. Thus, based on the geometry of a cylinder, where r is the radius of the cylinder, the area of imbibition is related to the volume of the swollen second composition as follows:

Aₚ=πr²+(2÷r)(Wp÷ρ)(1+(W_{H}÷W_{P})) (8)

The fluid imbibition rates into each composition are: A_{d}=A−Aₚ (9)
The fluid imbibition rates into each composition are: F=(k÷h)(A_{d}·Δπ_{d}) (10);
Q=(k÷h)(Aₚ·Δπₚ) (11), wherein k equals the osmotic permeability of the wall, h equals the wall thickness, Δπ_{d} and Δπ_{d} are the osmotic gradients for the first composition and the second composition, respectively. The total delivery rate, therefore, is equation (12):

dm÷dt=(k÷h)C{[A−πr²−(2÷r)(Wp÷ρ)(1+(W_{H}÷Wₚ))]Δπ_{d}+[πr²+(2÷r)(Wp÷ρ)(1+(W_{H}+Wₚ))]Δπ_{d}} (12)

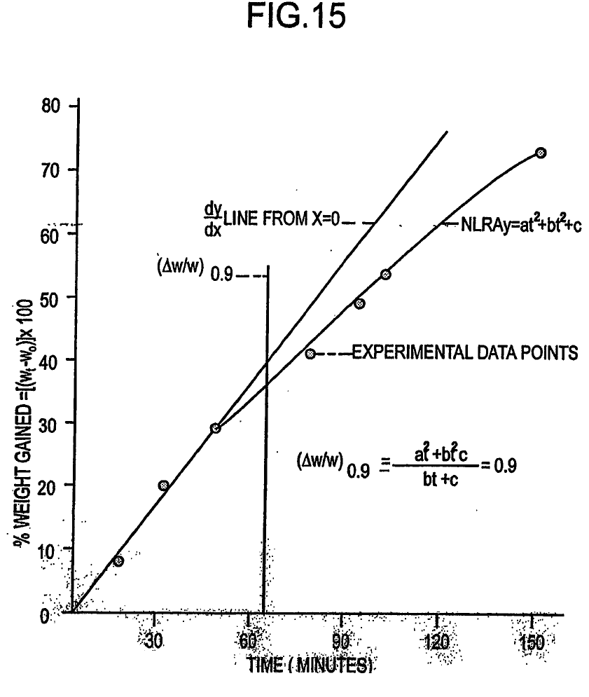

FIGS. 13 and 14 illustrate the osmotic dosage form in operation as described for FIGS. 11 and 12. In FIGS. 13 and 14, for osmotic dosage form 10, fluid is imbibed by the first composition at a rate determined by the permeability of the wall and the osmotic pressure gradient across the wall. The imbibed fluid continuously forms a composition comprising a proton pump inhibitor and the gel, which composition is released by the combined operations of the first composition and the second composition dosage form 10. These operations include the composition being osmotically delivered through the passageway due to the continuous formation of the composition, and by the swelling and increasing volume of the different second composition, is represented by the increase in height of the vertical lines in FIGS. 13 and 14. This latter swelling and increase in volume applies pressure against the first composition thereby aiding the first composition and simultaneously causing delivery of proton pump inhibitor through the osmotic passageway to the exterior of the dosage form. The dosage form can comprise more than one passageway, a passageway made as a microporous insert, or proton pump inhibitor releasing pores formed by leaching a leachable pore-former thereby providing pore-passageways for releasing the proton pump inhibitor to the exterior of the dosage form. Thus, the osmotic dosage form provided by this invention can be viewed as a single unit construction dosage form comprising two compositions containing two polymeric structures acting in concert for effective proton pump inhibitor administration to a patient. The dosage form 10 may comprise an enteric coating and/or polymer as exemplified in FIGS. 37-41.

The first composition and the second composition act together to substantially insure that delivery of the proton pump inhibitor from the compartment is controlled and constant over a prolonged period of time by two methods. First, the first composition imbibes external fluid across the wall, thereby forming a dispensable composition, which is substantially delivered at non-zero order rate, without the second composition present, since the driving force decays with time. Second, the second composition operating by imbibing external fluid across the wall continuously and, consequently, increases in volume as well as imbibition area, thereby exerting a force which can be constant, increasing or decreasing with time (depending on the osmotic formulation) against the first composition and diminishing the volume of proton pump inhibitor first composition, thus directing the proton pump inhibitor to the passageway at a controlled rate from the compartment. Additionally, as the first composition is squeezed out, that is, delivered from dosage form 10, the osmotic composition closely contacts the internal wall and generates a constant osmotic pressure and, therefore, effects a constant delivery rate in conjunction with the second composition. The swelling and expansion of the second composition, with its accompanying increase in volume, along with the simultaneous corresponding reduction in volume of the first composition, assures the delivery of proton pump inhibitor through the osmotic passageway at a controlled rate over time.

Dosage form 10 of FIGS. 11 through 14 can be made into many embodiments including the preferred embodiments for oral use for releasing a proton pump inhibitor in an intestinal tract. Oral system 10 can have various conventional shapes and sizes, such as round with a diameter of 3/16 inches to ⅝ inches. In these forms system 10 can be adapted for administering proton pump inhibitors to numerous patients, including humans.

The dosage forms 10 of FIGS. 11 through 14 can be used for delivering at least one proton pump inhibitor at a controlled rate. The osmotic dosage forms provide for delivery of the proton pump inhibitor at safe and effective amounts. While FIGS. 11 through 14 are illustrative of various dosage forms that can be made according to the invention, it is to be understood these dosage forms are not to be construed as limiting, as the dosage forms can take a wide variety of shapes, sizes and forms adapted for delivering proton pump inhibitors to the environment of use.

In accordance with the practice of the invention it has been found that dosage form 10 can be manufactured with a first composition and a different second composition mutually housed in cooperative relationship in the compartment of the dosage form. The compartment is formed by a wall comprising a material that does not adversely affect the proton pump inhibitor, osmagent, osmopolymer, and the like. The wall is permeable to the passage of an external fluid such as water and biological fluids, and it is substantially impermeable to the passage of proton pump inhibitors, osmagents, osmopolymers, and the like. The wall comprises a material that does not adversely affect an animal, or host, or the components comprising the dosage form, and the selectively semipermeable materials used for forming the wall are non-erodible and they are insoluble in fluids. Typical materials for forming the wall are, in one embodiment, cellulose esters, cellulose ethers and cellulose ester-ethers. These cellulosic polymers have a degree of substitution, D.S., on the anhydroglucose unit, from greater than 0 up to 3 inclusive. By degree of substitution is meant the average number of hydroxyl groups originally present on the anhydroglucose unit comprising the cellulose polymer that are replaced by a substituting group. Representative materials include cellulose acylate, cellulose diacylate, cellulose triacylate, cellulose acetate, cellulose diacetate, cellulose triacetate, mono-, di- and tricellulose alkanylates; mono-, di- and tricellulose aroylates, and the like. Exemplary polymers include cellulose acetate having a D.S. up to 1 and an acetyl content up to 21%; cellulose acetate having an acetyl content of 32% to 39.8%; cellulose acetate having a D.S. of 1 to 2 and an acetyl content of 21% to 35%; cellulose acetate having a D.S. of 2 to 3 and an acetyl content of 35% to 44.8%, and the like. More specific cellulosic polymers include cellulose propionate having a D.S. of 1.8 and a propyl content of 39.2% to 45% and a hydroxyl content of 2.8% to 5.4%; cellulose acetate butyrate having a D.S. of 1.8, an acetyl content of 13% to 15% and a butyryl content of 34% to 39%; cellulose acetate butyrate having an acetyl content of 2% to 29%, a butyryl content of 17% to 53% and a hydroxyl content of 0.5% to 4.7%; cellulose triacylates having a D.S. of 2.9 to 3 such as cellulose trivalerate, cellulose trilaurate, cellulose tripalmitate, cellulose trisuccinate, and cellulose triocta noate; cellulose diacylates having a D.S. of 2.2 to 2.6 such as cellulose disuccinate, cellulose dipalmitate, cellulose dioctanoate, cellulose dipentanoate, co-esters of cellulose such as cellulose acetate butyrate and cellulose acetate propionate, and the like.

Additional polymers include ethyl cellulose of various degree of etherification with ethoxy content of from 40% to 55%, acetaldehyde dimethyl cellulose acetate, cellulose acetate ethyl carbamate, cellulose acetate methyl carbamate, cellulose acetate dimethyl aminoacetate, semipermeable polyamides; semipermeable polyurethanes; semipermeable sulfonated polystyrenes; semipermeable cross-linked selective polymers formed by the coprecipitation of a polyanion and a polycation as disclosed in U.S. Pat. Nos. 3,173,876; 3,276,586; 3,541,005; 3,541,006, and 3,546,142; semipermeable polymers as disclosed in U.S. Pat. No. 3,133,132; semipermeable lightly cross-linked polystyrene derivatives; semipermeable cross-linked poly(sodium styrene sulfonate); semipermeable cross-linked poly(vinylbenzyltrimethyl ammonium chloride); water permeable membrane exhibiting a fluid permeability of 2.5×10⁻⁸ to 2.5×10⁴ (cm²/hr·atm) expressed per atmosphere of hydrostatic or osmotic pressure difference across the wall. The polymers are known to the art in U.S. Pat. Nos. 3,845,770; 3,916,899; and 4,160,020; and in Handbook of Common Polymers by Scott, J. R. and Roff, W. J., (1971), published by CRC Press, Cleveland, Ohio.

The expression, “passageway” comprises means and methods suitable for releasing the agent or drug from the osmotic system, as described herein. The passageways can optionally comprise enteric polymers.

One description of a passageway as the maximum and minimum dimensions for such a passageway, are disclosed in U.S. Pat. Nos. 3,845,770 and 3,916,899. The osmotically calibrated passageway has a maximum cross sectional area, Aₛ, defined by the relation (13) as follows: Aₛ(max)=(L/F)×(Qₚ/t)×(1/DS) (13); wherein L is the length of the passageway Qₚ/t is the mass delivery rate of the agent, D is the diffusion coefficient of the agent, S is the solubility of the agent in the fluid, and F is from 2 to 1000, said passageway having a minimum area Aₛ defined by relation (14) as follows: Aₛ(min)=[(Lv/t)×8×(πη/ΔP)]^{1/2} (14), wherein L is the length of the passageway, ν/t is the agent solution volume delivery rate, π is 3.14; η is the viscosity of agent solution or suspension dispensed from the dosage form and ΔP is the hydrostatic pressure difference between the inside and the outside of the compartment having a value up to 20 atmospheres. In addition, one or more passageways can be introduced into the dosage form. The number of passageways can be large, but should satisfy the condition that the delivery rate is substantially governed by the imbibition flux of water across the surrounding wall.

The passageway can be a pore formed by leaching sorbitol, and the like, from a wall, as disclosed in U.S. Pat. No. 4,200,098. This patent discloses pores of controlled size-porosity formed by dissolving, extracting or leaching a material from a wall, such as sorbitol from cellulose acetate. The pore-passageways extend from the inside to the outside of the wall for effective release of proton pump inhibitor to the exterior of the system. In U.S. Pat. No. 4,285,987 a composite delivery system is disclosed comprising a first dosage form that surround a second dosage form. The first comprises a cellulose acetate wall comprising leachable sorbitol for forming a pore for releasing osmotically active potassium chloride from an osmotic core. The second dosage form releases drug through a laser drilled passageway. The patent thereby discloses drug released through passageways formed by different techniques.

The osmotically effective compounds that can be used for the purpose of this invention include inorganic and organic compounds that exhibit an osmotic pressure gradient across a semipermeable wall against an external fluid. The osmotically effective compounds, along with the osmopolymers, imbibe fluid into the osmotic dosage form thereby making available in situ fluid for imbibition and/or absorption by an osmopolymer to enhance its expansion, and/or for forming a solution or suspension containing a proton pump inhibitor for its delivery through a passageway form the osmotic dosage form.

The osmotically effective compounds are known also as osmotically effective solutes, and also as osmagents. The osmotically effective compounds are used by mixing them with a proton pump inhibitor, or with an osmopolymer for forming a solution, or suspension containing the proton pump inhibitor that is osmotically delivered from the dosage form. The expression, “limited solubility” means the agent has a solubility of about less than 5% by weight in the aqueous fluid present in the environment. The osmotic solutes are used by homogeneously or heterogeneously mixing the solute with the proton pump inhibitor or osmopolymer and then charging them into the reservoir. The solutes and osmopolymers attract fluid into the reservoir producing a solution of solute in a gel which is delivered from the system concomitantly transporting undissolved and dissolved proton pump inhibitor to the exterior of the system. Osmotically effective solutes used for the former purpose include magnesium sulfate, magnesium chloride, potassium sulfate, sodium sulfate, lithium sulfate, potassium acid phosphate, d-mannitol, urea, inositol, magnesium succinate, tartaric acid, carbohydrates such as raffinose, sucrose, glucose, alpha-d-lactose monohydrate, sorbitol, and mixtures thereof. The amount of osmagent in the compartment will generally be from 0.01% to 30% or higher in the first composition, and usually from 0.01% to 40% or higher in the second composition.

The osmotic solute may be initially present in excess and it can be in any physical form that is compatible with the proton pump inhibitor, the dosage form, and the osmopolymer. The osmotic pressure of saturated solutions of various osmotically effective compounds and for mixtures of compounds at 37° C., in water, are listed in Table 1. In the table, the osmotic pressure π, is in atmospheres, atm. The osmotic pressure is measured in a commercially available osmometer that measures the vapor pressure difference between pure water and the solution to be analyzed and, according to standard thermodynamic principles, the vapor pressure ratio is converted into osmotic pressure difference. In Table 1, osmotic pressures of from 20 atm to 500 atm are set forth. Of course, the invention includes the use of lower osmotic pressures from zero, and higher osmotic pressures than those set forth by way of example in Table 1. The osmometer used for the present measurements is identified as Model 320B, Vapor Pressure Osmometer, manufactured by the Hewlett Packard Co., Avondale, Pa.

TABLE 1

| Compound or Mixture | Osmotic Pressure in atm. |
|---|---|
| Lactose-Fructose | 500 |
| Dextrose-Fructose | 450 |
| Sucrose-Fructose | 430 |
| Mannitol-Fructose | 415 |
| Sodium Chloride | 356 |
| Fructose | 355 |
| Lactose-Sucrose | 250 |
| Potassium Chloride | 245 |
| Lactose-Dextrose | 225 |
| Mannitol-Dextrose | 225 |
| Dextrose-Sucrose | 190 |
| Mannitol-Sucrose | 170 |
| Dextrose | 82 |
| Potassium Sulfate | 39 |
| Mannitol | 38 |
| Sodium Phosphate Tribasic 12 H₂O | 36 |
| Sodium Phosphate Dibasic 7 H₂O | 31 |
| Sodium Phosphate Dibasic 12 H₂O | 31 |
| Sodium Phosphate Dibasic Anhydrous | 29 |
| Sodium Phosphate Monobasic H₂O | 28 |

The osmopolymers suitable for forming the first proton pump inhibitor containing osmotic composition, and also suitable for forming the second drug free osmotic composition, are osmopolymers that exhibit fluid imbibition properties. The osmopolymers are swellable, hydrophilic hydrogel polymers which osmopolymers interact with water and aqueous biological fluids and swell or expand to an equilibrium state. The osmopolymers exhibit the ability to swell in water and retain a significant portion of the imbibed water within the polymer structure. The osmopolymers swell or expand to a very high degree, usually exhibiting a 2 to 50 fold volume increase. The osmopolymers can be noncross-linked or cross-linked. The swellable, hydrophilic polymers are, in one embodiment, lightly cross-linked, such cross-links being formed by covalent bonds, hydrogen bonds, ionic bonds or residue crystalline regions after swelling. The osmopolymers can be of plant, animal or synthetic origin. The osmopolymers are hydrophilic polymers. Hydrophilic polymers suitable for the present purpose include poly(hydroxy-alkyl methacrylate) having a molecular weight of from 30,000 to 5,000,000; anionic and cationic hydrogels; polyelectrolyte complexes; poly(vinyl alcohol) having a low acetate residual, cross-linked with glyoxal, formaldehyde, or glutaraldehyde and having a degree of polymerization from 200 to 30,000; a mixture of methylcellulose, cross-linked agar and carboxymethyl cellulose; a mixture of hydroxypropyl methylcellulose and sodium carboxymethylcellulose, hydroxypropyl-methylcellulose and sodium carboxymethyl cellulose; a water insoluble, water swellable copolymer reduced by forming a dispersion of finely divided copolymer of maleic anhydride with styrene, ethylene, propylene, butylene or isobutylene crosslinked with from 0.001 to about 0.5 moles of saturated cross-linking agent per mole of maleic anhydride in copolymer; water swellable polymers of N-vinyl lactams; polyoxyethylene-polyoxypropylene gel; polyoxybutylene-polyethylene block copolymer gel; carob gum, polyacrylic gel; polyester gel; polyurea gel; polyether gel; polyamide gel; polyimide gel; polypeptide gel; polyamino acid gel; polycellulosic gel; polygum gel; initially dry hydrogels that generally imbibe and absorb water which penetrates the glassy hydrogel and lowers its glass transition temperature, and the like.

Other osmopolymers include hydrogels such as Carbopol® acidic carboxy polymers, a polymer of acrylic acid crosslinked with a polyallyl sucrose, also known as carboxypolymethylene and carboxyvinyl polymer having a molecular weight of 250,000 to 4,000,000; Cyanamer® polyacrylamides; cross-linked water swellable indene-maleic anhydride hydrogel polymers; Good-rite® polyacrylic acid having a molecular weight of 80,000 to 200,000; Polyox® polyethylene oxide polymers having a molecular weight of 100,000 to 5,000,000 and higher; starch graft copolymers; Aqua-Keeps® acrylate polymer polysaccharides composed of condensed glucose units such as diester cross-linked polyglucan; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Company, Cleveland, Ohio. The amount of osmopolymer in the first composition is about 10% to 90%, and the amount of osmopolymer in the second composition is 20% to 100%, with the total weight of all ingredients in a composition equal to 100%. In one embodiment, the osmopolymer identified as P₁ comprising the first composition is different than the osmopolymer identified as P₂ comprising the second composition. The osmopolymer in the first composition can be structurally different than the osmopolymer in the second composition. Or, the osmopolymer's molecular weight in the second osmotic composition is larger than the molecular weight of the osmopolymer in the first composition. The osmopolymer P₁ comprising the first composition comprising the proton pump inhibitor serves as a pharmaceutically acceptable carrier for transporting the proton pump inhibitor from the dosage form in the form of a paste or gel-like ribbon, and it also contributes to the driving force that cooperates with osmopolymer P₂ comprising the second composition that delivers the proton pump inhibitor through the passageway from the dosage form. The phrase, “pharmaceutically acceptable carrier,” as used for the purpose of this invention, means the proton pump inhibitor is mixed with a gel and is transported with the gel from the dosage form. During operation of the dosage form fluid is imbibed into the dosage form resulting in the viscosity of P₂ being greater than the viscosity of P₁. In this operation P₁ and P₂ operate as a single unit substantially free of a void between their interfaced contacting surfaces of osmopolymer P₁ and P₂ for successful delivery of the proton pump inhibitor from the osmotic dosage form.

Osmopolymer fluid imbibition determination for a chosen polymer can be made by following the procedure described below. A round die having an inner diameter of ½ inch, fitted with a ½ inch diameter stainless steel plug, is charged with a known quantity of polymer with the plugs extending out either end. The plugs and the die were placed in a Carver press with plates between 200° F. and 300° F. A pressure of 10,000 to 15,000 psi was applied to the plugs. After 10 to 20 minutes of heat and pressure the electrical heating to the plates was turned off, and tap water circulated through the plates. The resulting ½ inch disks were placed in an air suspension coater charged with 1.8 kg saccharide cores, placebo cores, made of any sugar such as lactose, and so forth, and coated with cellulose acetate having an acetyl content of 39.8% dissolved in 94:6 w/w, CH₂Cl₂/CH₃OH, to yield a 3% w/w solution. The coated systems were dried overnight at 50° C. The coated disks were immersed in water at 37° C. and periodically removed for a gravimetric determination of water imbibed. The initial imbibition pressure was calculated by using the water transmission constant for the cellulose acetate, after normalizing imbibition values for membrane surface area and thickness. The polymer used in this determination was the sodium derivative of Carbopol-934® polymer, prepared according to the procedure of B. F. Goodrich Service Bulletin GC-36, “Carbopol® Water-Soluble Resins,” page 5, published by B. F. Goodrich, Akron, Ohio.

The cumulative weight gain values, y, as a function of time, t, for the water soluble polymer disk coated with the cellulose acetate were used to determine the equation of the line y=c+bt+at² passing through those points by at least square fitting technique.

The weight gain for the sodium salt of Carbopol-934® is given by the equation that follows: Weight gain equals 0.359+0.665t−0.00106t², wherein t is elapsed time in minutes. The rate of water flux at any time will be equal to the slope of the line that is given by the following equations (15) and (16):

dv/dt=[(d(0.359+0.665t−0.00106t²))/dt] (15)

dv/dt=0.665=0.00412t (16)

To determine the initial rate of water flux the derivative is evaluated at t=0, and dv/dt 0.665 μl/min, which is equal to the coefficient b. Then, normalizing the imbibition rate for time, membrane surface area and thickness, and the membrane permeability constant to water, Kπ may be determined according to the following equation (17):

Kπ=0.665 μl/min×(60 min/hour)×(1 ml/1000 μl)(0.008 cm/2.86 cm²) (17)

with Kπ=1.13×10⁻⁴ cm²/hr. The π value for NaCl was determined with a Hewlett Packard vapor pressure osmometer to be 345 atm±10%, and the K value for cellulose acetate used in this experiment calculated from NaCl imbibition values was determined to be 1.9×10⁻⁷ cm²/hr·atm.

Substituting these values into the calculated Kπ expression, (1.9×10⁻⁷/cm²/hr·atm) (π)=1.13×10⁻⁴ cm² hr gives π=600 atm at t=0. As a method for evaluating the efficiency of a polymer with respect to duration of zero order driving force, the percent of water uptake was selected before the water flux values decreased to 90% of their initial values. The value of the slope for the equation of a straight line emanating from the percent weight gained axis will be equal to the initial value of dy/dt evaluated at t=0, with the y intercept c defining the linear swelling time, with (dy/dt) 0=0.665 and the y intercept=0, which yields y=0.665t+0.359. In order to determine when the value of the cumulative water uptake is 90% below the initial rate, the following expression is solved for t:

0.9=(at²+bt+c)/(bt+c)=ΔW/w(0.9) (18)

(0.00106t²+0.665t+0.359)/(0.665t+0.359)=0.9 (19)

and solving for t,

−0.00106t²+0.0065t+0.0359=0

t=(−0.0665+[(0.0665)²−4(−0.00106)(0.0359)]½)÷[2(−0.00106)] (20)

t=62 min and the weight gain is −0.00106(62)²+(0.665)(62)+0.359 38 μl, with the initial sample weight=100 mg, thus (Δw/w) 0.9×100=38%. The results are presented in FIG. 15 for a graphical representation of the values. Other methods available for studying the hydrogel solution interface include rheologic analysis, viscometric analysis, ellipsometry, contact angle measurements, electrokinetic determinations, infrared spectroscopy, optical microscopy, interface morphology and microscopic examination of an operative dosage form.

The dosage form of the invention is manufactured by standard techniques. For example, in one embodiment the proton pump inhibitor is mixed with an osmagent and osmopolymer, and pressed into a solid possessing dimensions that correspond to the internal dimensions of the compartment adjacent to the passageway; or the proton pump inhibitor and other formulation forming ingredients and a solvent are mixed into a solid or a semisolid by conventional methods such as ballmilling, calendering, stirring or rollmilling, and then pressed into a preselected shape. Next, a layer of a composition comprising an osmagent and an osmopolymer is laced in contact with the layer of proton pump inhibitor composition, and the two layers surrounded with a semipermeable wall. The layering of the proton pump inhibitor composition and the osmagent/osmopolymer can be accomplished by conventional two layer tablet press techniques. The wall can be applied by molding, spraying, or dipping the pressed shapes into wall-forming materials. Another technique that can be used for applying the wall is the air suspension coating procedure. This procedure consists in suspending and tumbling the pressed compositions in a current of air and a wall forming composition until the wall surrounds and coats the two pressed compositions. They form a laminated wall. Finally, an enteric coating may be applied to the formulation/composition. The air suspension procedure is described in U.S. Pat. No. 2,799,241; J. Am. Pharm. Assoc., Vol. 48, pp 451 to 459 (1979); and, ibid, Vol. 49, pp 82 to 84 (1960). Other standard manufacturing procedures are described in Modern Plastics Encyclopedia, Vol. 46, pp 62 to 70 (1969); and in Pharmaceutical Science, by Remington, 14th Ed., pp 1626 to 1978 (1970), published by Mack Publishing Co., Easton, Pa.

Exemplary solvents suitable for manufacturing the wall, the laminates and laminae, include inert inorganic and organic solvents, that do not adversely harm the materials and the final wall or the final laminated wall. The solvents broadly include members selected from the group consisting of aqueous solvents, alcohols, ketones, esters, ethers, aliphatic hydrocarbons, halogenated solvents, cycloaliphatics, aromatics, heterocyclic solvents, and mixtures thereof. Typical solvents include acetone, diacetone alcohol, methanol, ethanol, isopropyl alcohol, butyl alcohol, methyl acetate, ethyl-acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, chloroform, nitroethane, nitropropane, tetrachloroethane, ethyl ether, isopropyl ether, cyclohexane, cyclo-octane, benzene, toluene, naphtha, 1,4-dioxane, tetrahydrofuran, diglyme, aqueous and nonaqueous mixtures thereof, such as acetone and water, acetone and methanol, acetone and ethyl alcohol, methylene dichloride and methanol, and ethylene dichloride and methanol.

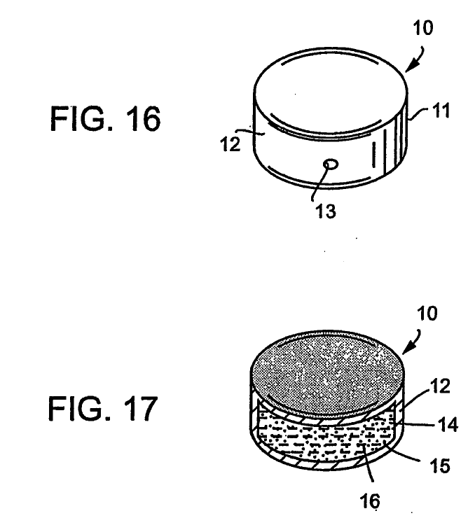

Another embodiment of the invention is represented by reference to FIGS. 16 and 17. The dosage forms exemplified in FIGS. 16-17 may further comprise an enteric coating or polymer as described for example, in FIGS. 37-41.

One example of an osmotic dosage form 10 is indicated in FIG. 16. In FIG. 16, osmotic dosage form 10 comprises a body 11 having a wall 12 and a passageway 13 in wall 12. Passageway 13 connects the interior with the exterior of the dosage form.

In FIG. 17, dosage form 10 of FIG. 16 is seen in opened-section with a portion of wall 12 removed for illustrating the internal structure of dosage form 10. Dosage form 10 comprises wall 12 that surrounds and defines an internal compartment 14. Passageway 13, seen in FIG. 16, connects compartment 14 with the exterior of dosage form 10. Compartment 14 contains a proton pump inhibitor 15 that in one embodiment (a) is soluble in an external fluid 16 that enters compartment 14 and it exhibits an osmotic pressure gradient across wall 12 against the external fluid, or in another embodiment (b) has limited solubility or is substantially insoluble in external fluid 16 and is mixed with an osmotically effective compound that is soluble in external fluid and exhibits an osmotic pressure gradient across the wall against the external fluid. Compartment 14 can contain also other compounds such as a surfactant for wetting the agent.

Wall 12 of osmotic dosage form 10 is a composite comprising at least two wall forming materials blended to form a semipermeable wall 12. Composite wall 12 is (a) substantially impermeable to the passage of an external fluid, (b) substantially impermeable to the passage of the proton pump inhibitor 15 and other compounds housed in compartment 14, (c) substantially inert in the presence of the proton pump inhibitor, salts thereof and/or solutions thereof, (d) maintains its physical and chemical integrity in the environment of use during the dispensing of the proton pump inhibitor, and (e) is non-toxic and made with non-toxic solvents.

In operation in the environment of use, dosage form 10 releases the proton pump inhibitor 15 housed in compartment 14 by fluid being imbibed into compartment 13 in a tendency towards osmotic equilibrium at a rate controlled by the permeability of wall 12 and the osmotic pressure gradient across wall 12 to continuously dissolve the proton pump inhibitor 15 which is osmotically pumped from dosage form 10 through passageway 13 at a controlled and continuous rate over a prolonged period of time. Dosage form 10, in another embodiment releases proton pump inhibitor 15 that has limited solubility in the fluid and is mixed with an osmotically effective compound by fluid being imbided through wall 12 into compartment 14 in a tendency towards osmotic equilibrium at a rate controlled by the permeability of wall 12 and the osmotic gradient across wall 12 to continuously dissolve the osmotic effectively compound to form a solution containing the proton pump inhibitor which is pumped from dosage form 10 through passageway 13 at a controlled and continuous rate over a prolonged period of time.

Dosage form 10 of FIGS. 16 and 17 can be made in many embodiments including the embodiment for oral use, that is, for releasing in the intestinal tract at least one proton pump inhibitor over an extended period of time. Oral dosage form 10 can have various conventional shapes and sizes such as round with a diameter of 3/16 inch to ⅝ inch, or more, or it can be shaped like a capsule having a range of sizes from triple zero to zero, and from 1 to 8.

In accordance with the practice of this invention, it has been found osmotic dosage form 10 can be manufactured with an improved wall 12 comprising at least two wall forming materials that act together to yield a semipermeable wall that operates like a wall formed of a single material. Wall 12 comprises a primary wall forming material comprising a selectively permeable cellulose ether that is permeable to the passage of fluid and substantially impermeable to the passage of proton pump inhibitors. The preferred cellulose ether is ethyl cellulose. Ethyl cellulose is a non-toxic polymer, insoluble in water, essentially insoluble in the digestive system, soluble in the organic solvent ethyl alcohol and in solvent systems consisting essentially of alcohol and water. A more preferred ethyl cellulose has an ethoxy group degree of substitution of 1.5 to 3 about 40 to 50% ethoxy content; and a viscosity range of 7 to 100 centipose, or higher.

Semipermeable wall 12 contains also a wall forming pharmaceutically acceptable water insoluble polymer, or a pharmaceutically acceptable water soluble polymer or a pharmaceutically acceptable water soluble agent. These polymers or agents, in either embodiment, are permeability enhancers that aid in regulating the passage of fluid into the osmotic dosage form. Exemplary water soluble polymers include celluloses (e.g., hydroxypropyl cellulose, hydroxypropyl methylcellulose, hydroxyethyl cellulose, ethyl methylcellulose, methylcellulose), acrylics (e.g., polyacrylic acid, polyethyl methacrylate, polymethyl methacrylate, pyrrolidones including polyvinyl pyrrolidone, alkylated vinylpyrrolidone polymers, poly(vinyl-pyrrolidone/vinyl acetate)copolymers, vinylpyrrolidone/dimethylamino-ethylmethacrylate copolymers), maleic acid polymers (e.g., monobutyl ester of poly(methyl vinylether/maleic acid), monoethyl ester of poly(methylvinyl ether/maleic acid), poly(methyl vinylether/maleic anhydride)copolymer, polyvinyl alcohol hydrolyzed 75 to 85%). Exemplary water soluble agents include polyethylene glycol, polyethylene oxide, guar gum, gum arabic, dextran, citric acid, triethyl citrate, acetyl-triethyl citrate, sucrose, fructose, glycerin, triacetin, and the like.

Exemplary water insoluble, alcohol-water soluble or dispersable polymers include carboxy polymers, blended with hydroxy polymers and insolubilized by curing with an energy source. The preferred carboxy polymers is carboxyvinyl polymer, also known as carboxypolymethylene, a polymer consisting of acrylic acid crosslinked with polyallyl sucrose as described in U.S. Pat. Nos. 2,798,053 and 2,909,462 as sold under the trademark Carbopol®. The preferred hydroxy polymer is hydroxypropyl cellulose sold under the trademark Klucel®. The preferred ratio of these is polyhydroxy:polycarboxy 4:1. Other carboxy polymers can optionally be used including poly(methyl vinylether maleic anhydride), ethylene/acrylic acid copolymer, ethylene maleic acid anhydride copolymers, methacrylic acid ethylacrylate copolymers, and the like. Other hydroxy polymers include hydroxyethyl cellulose, hydroxyethyl starch, poly(hydroxyethyl methacrylate), hydroxybutyl methylcellulose, and the like. Other water-insoluble, alcohol-water soluble polymers include cellulose nitrate, polyalkyds, polyvinyl acetal, polyvinyl butyral, vinyl alcohol-vinyl acetate copolymer, vinyl alcohol-vinyl butyral copolymer, polyethylacrylate and the like.

The energy source used in this process can be heat, electromagnetic radiation such as ultraviolet light, microwave irradiation, heat with irradiation, heat with forced air, vacuum treatment, ultrasonic vibration, and the like. The preferred energy source is heat with vacuum which serves to insolubilize the polymer. The product of this reaction is the ester crosslinked polymer which is hydrophilic and substantially insoluble in biological fluids. The water byproduct is continuously removed as the reaction proceeds.

Optionally, water insoluble agents can be included as plasticizers into the wall to increase its flexibility. Agents in this group include tributyl citrate, acetyltributyl citrate, acetyl-tri-2-ethylhexayl citrate, tributyl sebeccate, castor oil, castor oil fatty acids, mono-, di-, and triglycerides, and oils such as corn, cottonseed, peanut and soya.

The term composite as used herein means the wall is comprised of a blend of these materials that act together to form the operative semipermeable wall of the dosage form. The amount of the (a) primary wall forming cellulose ether present in the wall is about 20 to about 90 wt %/, weight percent, the amount of (b) water soluble hydrophilic polymer or hydrophilic agent is about 10 to about 50 wt %, the amount of (c) water insoluble, hydrophilic polymer is about 10 to about 80 wt %, or, (d) the composite wall consists of (a) and a mixture of (b) and (c), with the semipermeable composite wall comprising 100 wt %. Polymers (b) and (c) are formulated into the semipermeable wall to (e) provide a more uniform rate of release, to (f) provide a more complete release of the proton pump inhibitor, to (g) impart physical strength and prolong the life of the wall, and to (h) provide a means for adjusting the permeability of the wall by selecting the ratio of (a) to (b) or (c), or the ratio of (a) to (b) and (c). The permeability change is effected by corresponding change in the proportions and it is reproducible. The polymers are known to the art in Handbook of Common Polymers, by Scott and Roff, 1971, published by CRC Press; in Materials Handbook, by Brady and Clauser, 1977, published by McGraw-Hill; and in Handbook of Plastics and Elastomers, by Harper, 1975, published by McGraw-Hill.

Exemplary solvent systems useful for manufacturing the semipermeable wall of the osmotic dosage form are in a presently preferred embodiment non-toxic solvent systems. These systems include ethyl alcohol, and blends of ethyl alcohol with water such as ethanol, ethanol-water (95:5 wt:wt), ethanol-water (90:10), ethanol-water (70:30), and the like.

The expression passageway as used herein comprises means and methods suitable for releasing the proton pump inhibitor from the dosage form. The expression includes aperture, orifice, or bore through the wall formed by mechanical procedures, or by eroding an erodible element, such as a gelatin and/or enteric polymer plug, in the environment of use. A detailed description of osmotic passageways and the maximum and minimum dimensions for a passageway are disclosed in U.S. Pat. Nos. 3,845,770 and 3,916,899.

The osmotic dosage forms of the invention are manufactured by standard techniques. For example, in one embodiment the proton pump inhibitor and other ingredients that may be housed in the compartment and an optional solvent are mixed into a solid, semi-solid or gel form by conventional methods such as ballmilling, calendering, stirring or rollmilling, the solvent evaporated, and then pressed into a preselected shape. The wall forming the dosage form can be applied by molding, spraying, dipping, or pan coating the pressed shape into wall forming materials. In another embodiment a wall can be cast into a film, shaped to the desired dimensions, sealed to define a hollow compartment that is filled with proton pump inhibitor, and then closed with a passageway formed in the wall. The dosage form also can be manufactured with an empty compartment that is filled through the passageway. High frequency electronic techniques can be used to provide dosage forms with wall having clean edges. Another, and presently preferred technique that can be used is the air suspension procedure. This procedure consists in suspending and tumbling the pressed agent and other ingredients in a current of air and the wall forming compositions until the wall is applied to the agent. The air suspension procedure is described in U.S. Pat. No. 2,799,241; in J. Am. Pharm. Assoc., Vol. 48, pages 451 to 459, 1959; and ibid, Vol. 49, pages 82 to 84, 1960. Other standard manufacturing procedures are described in Modern Plastics Encyclopedia, Vol. 46, pages 62 to 70, 1969; in Pharmaceutical Sciences, by Remington, 14th Ed., pages 1626 to 1678, 1970; and in U.S. Pat. No. 4,236,525.

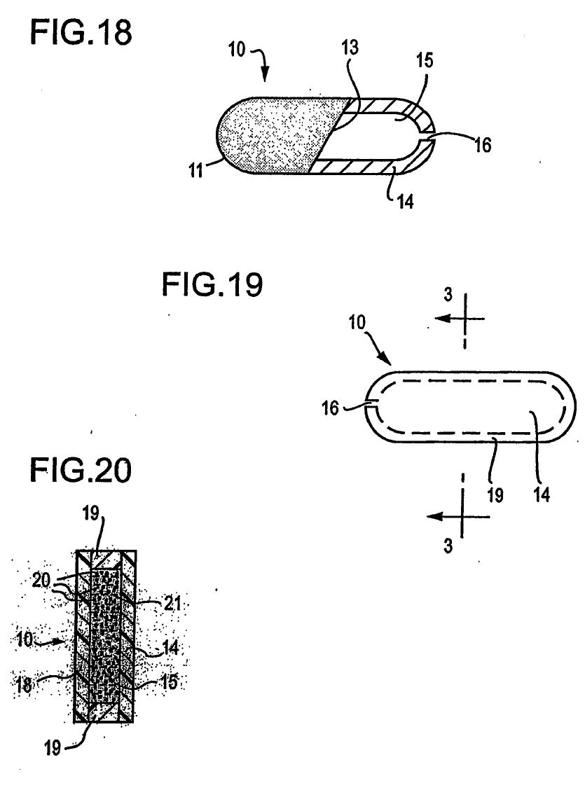

FIGS. 18-20 describe another embodiment of the invention. The dosage forms exemplified in FIGS. 18-20 may further comprise an enteric coating or polymer as described for example, in FIGS. 37-41.

In FIG. 18, dosage form 10 is comprised of a body portion 11. Dosage form 10 is shown in open section 13 and dosage form 10 is comprised of a wall 14 surrounding a compartment 15. Compartment 15 is a means for containing a composition comprising at least one proton pump inhibitor, not shown in FIG. 18. The dosage form 10 has at least one passageway 16 that communicates with compartment 15 and the exterior of the dosage form 10.

Wall 14 of dosage form 10 is comprised in total or in at least a part of a semi-permeable membrane that possesses permeability to an external fluid while simultaneously being essentially impermeable to the proton pump inhibitor composition housed in compartment 15. That is, body 11 formed of wall 14 can be of unit construction, or composite construction with a section of a semi-permeable membrane either formed integral in wall 14, or optionally lined or laminated to wall 14. Wall 14 can be formed of a semi-permeable material that has uniform properties across all its dimensions, that is, it is substantially imperforate or substantially homogenous, or wall 14 can be formed of a material that is microporous, that is, a material having micropores or microholes, or it can be a semi-permeable material possessing both of these properties while remaining essentially impermeable to a product present in compartment 15. In operation, when wall 14 is comprised of a material that is substantially imperforate, molecules of the external fluid dissolve in and diffuse through wall 14 by the process of diffusion into compartment 15. When wall 14 is made from a microporous material, molecules of external fluid migrate and diffuse into the micropores, as by diffusion, then into compartment 15. When wall 14 is made from semi-permeable material having both of these properties, external fluid enters the chamber by a concurrent operation of each of these mechanisms, that is, by diffusion through wall 14 and by diffusion through the pores of wall 14. Wall 14 is formed of synthetic or naturally occurring semipermeable materials and a detailed description of these materials appears later in this specification.

In FIG. 19 there is seen another dosage form 10. Dosage form 10 in this embodiment is an oral dosage form and it is illustrated in FIG. 19 in top perspective view. Dosage form 10 comprised of a wall 14 formed of a material that is permeable to an external fluid but substantially impermeable to a drug, not seen in FIG. 19 that is housed in dosage form 10. Wall 14 carries on its inner surface an inner positioned wall 19 formed with a passageway 16, schematically illustrated by dashed lines, which wall 19 is extended around the perimeter of wall 14 to engage it in sealed relation with another wall, not shown in FIG. 19 and positioned distant from wall 14. The distant wall can be of the same construction as wall 14 or it can be formed of a material that is optionally permeable to an external fluid and impermeable to the proton pump inhibitor to form a composite orally administered extended release dosage form.

Referring to FIG. 20, oral dosage form 10 is seen in cross-section through 3-3 of FIG. 19. Oral dosage form 10 of FIG. 20 is comprised of a first wall 14 and a third wall 18 distant from first wall 14. Wall 14 and wall 18 bear on their inner surface a second wall 19 that extends around the outer perimeter of wall 14 and wall 18 to form a closed drug compartment 15. Drug compartment 15 is comprised of a composition comprising at least one proton pump inhibitor 20 and, optionally, pharmaceutically acceptable carriers. A passageway, not seen in FIG. 20, communicates with drug chamber 15 and the exterior of the dosage form 10 for the release of the proton pump inhibitor 20. Wall 14 and wall 18 can be the same or they can be different and at least one of the walls, 14 or 18, or both of the walls, is comprised of a semi-permeable material permeable to the passage of external fluid 21, for example, intestinal fluids, by diffusion, or at least one of the walls, 14 or 18, is comprised of a microporous material into which intestinal fluid can permeate to subsequently enter chamber 15, as by diffusion. While at least one of wall 14 or wall 18 is permeable to intestinal fluids 21, both of the walls are essentially impermeable to the passage of the proton pump inhibitor 20. Wall 19 of dosage form 10 is formed of a non-allergenic, biologically inert, insoluble in intestinal fluid material suitable for joining wall 14 and wall 18 together to form an essentially closed compartment 15 as defined by the inner surfaces of walls 14, 18 and 19. Dosage form 10 when made from a material that is insoluble in intestinal fluids naturally passes through the intestinal tract or, dosage form 10 can be made from a bioerodible material that bioerodes in situ to harmless end products after the dosage form has completed its predetermined proton pump inhibitor release program. The walls, 14, 18 and 19 of dosage form 10 of the invention are formed of a material that can be rigid, semi-rigid, semi-flexible, flexible or the like.

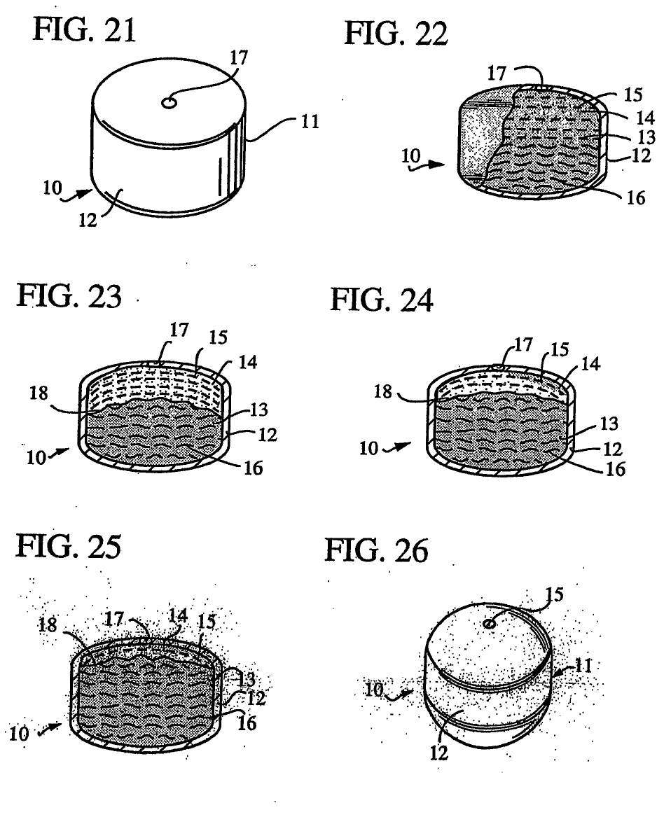

In another embodiment, the invention is represented by reference to FIGS. 21-25. The invention relates to an osmotic dosage form comprising a semipermeable wall surrounding a compartment containing a proton pump inhibitor, and a layer of a water-swellable cross-linked hydrogel driving member. A passageway through the wall connects the exterior of the dosage form with the proton pump inhibitor for delivering the proton pump inhibitor from the dosage form. The dosage form 10 may optionally comprise enteric coatings or polymers as exemplified in FIGS. 37-41.

In FIGS. 21 through 25, dosage form 10 is seen comprised of a body member 11 having a wall 12 that surrounds and forms a compartment 13, as seen in opened dosage form 10 in FIGS. 22 through 25. Compartment 13, comprises a layer of a proton pump inhibitor and is identified by dots 14, which proton pump inhibitor when soluble in the fluid exhibits an osmotic pressure gradient across wall 12 against an exterior fluid, indicated by dashes 15, that is imbibed into compartment 13. Compartment 13 in another embodiment contains a layer of proton pump inhibitor 14 that has limited solubility or is substantially insoluble in fluid 15, and it exhibits a limited, or it may not exhibit any osmotic pressure gradient across wall 12 against the exterior fluid. When the proton pump inhibitor 14 has a limited solubility, or if it is substantially insoluble in fluid 15, it can be mixed with an osmagent that is soluble in the external fluid and exhibits an osmotic pressure gradient across wall 12 against the fluid. Wall 12 is formed of a polymeric material that is substantially permeable to the passage of the external fluid, and it is substantially impermeable to the passage of agent and osmagent. The semipermeable polymer forming wall 12 is non-toxic and it maintains its physical and chemical integrity during the life of dosage form 10. Typical materials for forming wall 12 include semipermeable polymers known to the art as osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethylaminoacetate, cellulose acetate ethyl carbamate, cellulose acetate chloroacetate, cellulose dipalmatate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142, semipermeable polymers as disclosed by Loeb and Sourirajan in U.S. Pat. No. 3,133,132, lightly cross-linked polystyrene derivatives, cross-linked poly(sodium styrene sulfonate), poly(vinylbenzyltrimethyl ammonium chloride), cellulose acetate having a degree of substitution up to 1 and an acetyl content up to 21%, cellulose diacetate having a degree of substitution of 1 to 2 and an acetyl content of 21 to 35%, cellulose triacetate having a degree of substitution of 2 to 3 and an acetyl content of 35 to 44.8%, as disclosed in U.S. Pat. No. 4,160,020.

Compartment 13 further houses a layer of an expandable driving member made from a hydrogel and identified by wavy lines 16. Hydrogel 16 is a hydrophilic, water insoluble polymer, optionally cross-linked, which possesses osmotic properties such as the ability to imbibe external fluid and exhibit an osmotic pressure gradient across the semipermeable wall against the fluid. Hydrophilic polymeric materials for the purpose include poly(hydroxyalkyl methacrylate), poly(N-vinyl-2-pyrrolidone), anionic and cationic hydrogels, polyelectrolyte complexes, poly(vinyl alcohol) having a low acetate residual and cross-linked with glyoxal, formaldehyde, or glutaraldehyde, methyl cellulose cross-linked with dialdehyde, a mixture of cross-linked agar and carboxymethyl cellulose, a water insoluble, water-swellable copolymer produced by forming a dispersion of finely divided copolymer of maleic anhydride with styrene, ethylene, propylene, butylene, or isobutylene cross-linked with from 0.001 to about 0.5 moles of a polyunsaturated cross-linking agent per mole of maleic anhydride in the copolymer, water-swellable polymers of N-vinyl lactams, cross-linked polyethylene oxides, and the like. Other hydrogels include hydrogels exhibiting a cross-linking of 0.05 to 60%, hydrophilic hydrogels known as Carbopol®& acidic carboxy polymer, Cyanamer®& polyacrylamides, cross-linked water-swellable indene-maleic anhydride polymers, Good-rite® polyacrylic acid, polyethyleneoxide, starch graft copolymers, Aqua-Keeps® acrylate polymer, diester cross-linked polyglucan, and the like. The hydrogels are known to the prior art in U.S. Pat. No. 3,865,108 issued to Hartop; in U.S. Pat. No. 4,002,173 issued to Manning; in U.S. Pat. No. 4,207,893 issued to Michaels; and in Handbook of Common Polymers by Scott and Roff, published by the Chemical Rubber Company, Cleveland, Ohio. Hydrogel 16 absorbs fluid imbibed into the compartment and swells or expands to some equilibrium state. At equilibrium the osmotic pressure of the hydrogel approximately equals the swelling pressure of the hydrogel, and the osmotic pressure of the hydrogel network is the driving force of the swelling, expanding member 16. Hydrogel 16 is in contact with the proton pump inhibitor 14 and, at the interface formed by the hydrogel and the proton pump inhibitor, a thin precipitate 18 forms in the outer surface of hydrogel 16. The precipitate forms in the presence of a solution containing the proton pump inhibitor, or the proton pump inhibitor and an osmagent, and it is substantially impervious and restricts the passage of the proton pump inhibitor 14 into hydrogel 16. The precipitate further serves as an in situ formed membrane integral with the hydrogel for applying pressure against the proton pump inhibitor 14 during operation of dosage form 10. The osmagent present in the dosage form are osmotically effective compounds soluble in fluid that enter the dosage form, and exhibit an osmotic pressure gradient across the semipermeable wall against the exterior fluid. Osmotically effective osmagents useful for the present purpose include magnesium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol inositol, raffinose, sucrose, glycose, mixtures thereof, and the like.

Dosage form 10 releases the proton pump inhibitor 14 through a passageway 17 in wall 12 that communicates with the proton pump inhibitor 14 with the exterior of dosage form 10. Dosage form 10 releases the proton pump inhibitor 14 by fluid being imbibed into compartment 13 in a tendency towards osmotic equilibrium at a rate determined by the permeability of wall 12 and the osmotic pressure gradient across wall 12. The imbibed fluid continuously forms a solution containing the proton pump inhibitor, or a solution of osmagent containing the proton pump inhibitor, in suspension which solution in either instance is released by the combined operation of dosage form 10. These operations include the solution being osmotically delivered through passageway 17 due to the continuous formation of solution in the compartment, and by the hydrogel swelling and increasing in volume and applying pressure against the solution thereby delivering it to the exterior of dosage form 10.

Compartment 13 operates to substantially insure that delivery of the proton pump inhibitor 14 from compartment 13 is constant over an extended period of time by two methods. First, hydrogel 16 operates to continuously concentrate the proton pump inhibitor 14 by imbibing some fluid from the proton pump inhibitor 14 to keep the concentration of the proton pump inhibitor 14 from falling below saturation. Secondly, hydrogel 16 by imbibing external fluid 15 across wall 12 continuously increases its volume, as illustrated by the expansion of hydrogel 16 in FIGS. 23 through 25, thereby exerting a force on the proton pump inhibitor 14 and diminish the volume of the proton pump inhibitor 14, thusly concentrating the proton pump inhibitor 14 in compartment 13. The swelling and expansion of hydrogel 16, with its accompanying increase in volume, along with the simultaneous, corresponding reduction in volume of the proton pump inhibitor 14, assures the delivery of the proton pump inhibitor 14 at an extended rate over time.

Dosage form 10 of FIGS. 21 through 25 can be made into many embodiments including the presently preferred embodiments for oral use, that is, for releasing either a locally or systemically acting therapeutic proton pump inhibitor in the intestinal tract over time. Oral system 10 can have various conventional shapes and sizes such as round with a diameter of 3/16 inches to ½ inch, or it can be shaped like a capsule having a range of sizes from triple zero to zero, and from 1 to 8.

In another embodiment, the invention is represented by reference to FIGS. 26-33. The invention relates to an osmotic dosage form that comprises a semipermeable wall surrounding a proton pump inhibitor compartment and an osmagent compartment separated from each other by an expandable film. The osmagent compartment can increase its volume while correspondingly diminishing the volume of the proton pump inhibitor compartment, thereby improving the delivery kinetics of the dosage form and the amount of the proton pump inhibitor released from the dosage form over a prolonged period of time. If necessary to prevent release of the proton pump inhibitor into the acidic stomach, the dosage form may be coated with an enteric coating layer or with an enteric polymer, as exemplified in FIGS. 37-41.

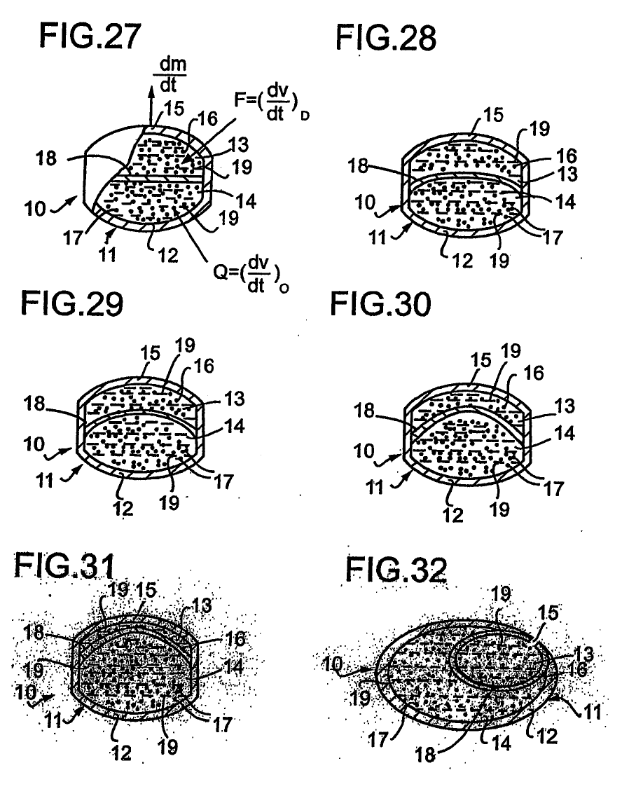

In FIGS. 26 through 31, dosage form 10 is comprised of a body 11 having a wall 12 that surrounds and forms a first compartment 13 and a second compartment 14, illustrated in FIGS. 27 through 31 in cross section, and a passageway 15 that communicates with compartment 13 and the exterior of dosage form 10. Typical materials for forming wall 12 include semipermeable materials known to the art as osmosis and reverse osmosis membranes such as cellulose acetate, cellulose triacetate, agar acetate, amylose triacetate, beta glucan acetate, cellulose diacetate, acetaldehyde dimethyl acetate, cellulose acetate ethyl carbamate, polyamides, polyurethane, sulfonated polystyrenes, cellulose acetate phthalate, cellulose acetate methyl carbamate, cellulose acetate succinate, cellulose acetate dimethylaminoacetate, cellulose acetate ethyl carbamate, cellulose acetate chloroacetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, selectively permeable polymers formed by the coprecipitation of a polycation and a polyanion as disclosed in U.S. Pat. Nos. 3,173,876; 3,276,586; 3,541,005; 3,541,006; and 3,546,142. Compartment 13, as seen in FIG. 27, in one embodiment contains a proton pump inhibitor 16 that is soluble to very soluble in an external fluid 19, exhibits an osmotic pressure gradient across wall 12 against the fluid and is in direct communication with wall 12, or compartment 13 in another embodiment contains a proton pump inhibitor 16 that has limited solubility or is substantially insoluble in fluid 19 and exhibits a limited, or it not exhibit any, osmotic pressure gradient across wall 12 against the fluid, and is in contact with the interior surface of semipermeable wall 12. When the proton pump inhibitor 16 has limited solubility or it is substantially insoluble in fluid 19, it can be mixed with an osmagent that is soluble in the external fluid and exhibits an osmotic pressure gradient across wall 12 against the fluid.

Compartment 14, as seen in FIG. 27, in one embodiment contains an osmagent 17, which is an osmotically effective compound, that is soluble in fluid 19 and exhibits an osmotic pressure gradient across wall 12 against fluid 19, or compartment 14 can contain a plurality of osmagents 17 with each exhibiting the same or different osmotic pressure gradients across wall 12 against fluid 19. Osmotically effective compounds 17 useful for the present purpose include magnesium sulfate, magnesium chloride, sodium chloride, lithium chloride, potassium sulfate, sodium carbonate, sodium sulfite, lithium sulfate, potassium chloride, calcium carbonate, sodium sulfate, calcium sulfate, potassium acid phosphate, calcium lactate, d-mannitol, urea, inositol, magnesium succinate, tartaric acid, carbohydrates such as raffinose, succrose, glycose, and mixtures thereof. Osmagent 17 suitable for housing in compartment 14 also includes starches and carbohydrates such as algin, sodium alginate, potassium alginate, carrageenan, fucoridan, furcellaran, laminaran, hypnea, gum arabic, gum ghatti, gum karaya, locust bean gum, pectin, starch, mixtures thereof, and the like.

Compartments 13 and 14 of dosage form 10 are separated by a contiguous film or membrane 18, formed from above materials and seen in FIGS. 27 through 31, for improving and assisting in regulating delivery and the amount of the proton pump inhibitor 16 from compartment 13. Film 18 is free of passageways and it is formed of an expandable material that can move from an initial or rested position, seen in FIG. 27, through a series of sequential changes as seen in FIGS. 28 through 30, to form fully expanded film 18 as seen in FIG. 31.

Compartment 14 operates in cooperation with system 10, particularly compartment 13, to release the proton pump inhibitor 16 to the environment of use. Dosage form 10 in one embodiment, releases proton pump inhibitor 16 in compartment 13 and soluble in the external fluid by fluid being imbibed into compartment 13 in a tendency towards osmotic equilibrium at a rate controlled by the permeability of wall 12 and the osmotic pressure gradient across wall 12 to dissolve proton pump inhibitor 16 which is osmotically pumped from system 10 through passageway 15 over a prolonged period of time. Compartment 14 operates to substantially insure that delivery of proton pump inhibitor 16 from compartment 13 is constant over an extended period of time by two methods. First, compartment 14 operates to continuously concentrate proton pump inhibitor 16 by imbibing fluid from compartment 13 through film 18 to keep the concentration of proton pump inhibitor 16 from falling below saturation. Secondly, compartment 14 by imbibing external fluid 19 across wall 12 continuously increases its volume, thereby exerting a force on film 18 urging it to expand into and diminish the volume of compartment 13, thusly insuring continuous saturation of proton pump inhibitor 16 in compartment 13. FIGS. 28 through 31 illustrate the expansion of film 18 with the accompanying increase in volume of compartment 14 along with the simultaneous, corresponding reduction in volume of compartment 13. Compartment 13 can contain various amounts of proton pump inhibitor 16. Proton pump inhibitor 16 can be present in large amounts as a solid, which is mixed with fluid imbibed into compartment 13 to form a solution or suspension for release from dosage form 10. In this manner, compartment 13 operates as a formulation compartment and thereby makes possible (a) the housing of large amounts of proton pump inhibitor and (b) increases the amount of proton pump inhibitor delivered at an extended rate from dosage form 10. Dosage form 10, in another embodiment, releases proton pump inhibitor 16 that has limited solubility in the fluid and is mixed with an osmagent by fluid being imbibed through wall 12 into compartment 13 in a tendency towards osmotic equilibrium at a rate controlled by the permeability of wall 12 and the osmotic pressure gradient across wall 12 to continuously dissolve the osmagent and form a solution containing proton pump inhibitor 16 that is pumped from system 10 through passageway 15. In this embodiment, compartment 14 operates as described above. In other embodiments, proton pump inhibitor 16 can be present as a gel, paste or semi-solid which formulation is released by the compartments of the system operating as a unit system as described above.

Wall 12 of dosage form 10 is comprised of a semipermeable material that is permeable to the passage of an external fluid and it is essentially impermeable to proton pump inhibitor 16, osmagent 17, and other ingredients housed in compartments 13 and 14. Film 18 of dosage form 10 is formed of a material that is deformable, either permeable or impermeable to the passage of fluid, and in both instances, impermeable to the passage of proton pump inhibitor and osmagent; and, it can undergo expansion over a prolonged period of time. Wall 12 and film 18 can be formed of synthetic or naturally occurring materials.

Dosage form 10 of FIGS. 26 through 31 can be made into many embodiments including the present embodiments for oral use, that is, for releasing either a locally or systemically acting therapeutic proton pump inhibitor in the intestinal tract over an extended period of time. Oral dosage form 10 can have various conventional shapes and sizes such as round with a diameter of 3/16 inch to ½ inch, or it can be shaped like a capsule having a range of sizes from triple zero to zero, and from 1 to 8

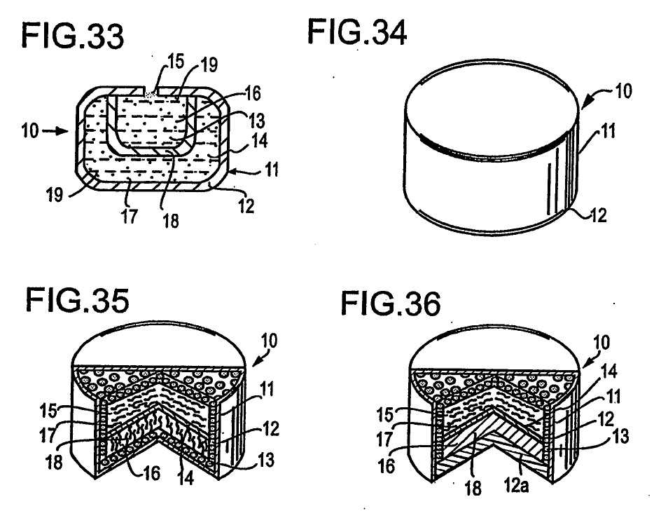

FIGS. 32 and 33 represent additional embodiments of dosage form 10 manufactured according to the invention and designed for dispensing proton pump inhibitor 16 to numerous environments of use. If necessary to prevent release (and subsequent degradation) of the proton pump inhibitor into the acidic stomach, the dosage form 10 may be coated with an enteric coating layer or comprise an enteric polymer, as described in FIGS. 37-41.

In FIGS. 32 and 33, dosage form 10 is seen in opened section and it is similar to dosage form 10 of FIGS. 26 through 31, with each dosage form comprising a body 11 having a wall 12 that surrounds a proton pump inhibitor compartment 13 and an osmagent compartment 14, with compartment 13 having a passageway 15 that communicates with the exterior of dosage form 10. Compartments 13 and 14 both house the ingredients housed in FIGS. 27 through 31. In FIGS. 32 and 33, compartments 13 and 14 are separated by movable film 18 that forms the entire barrier member between the compartments. Dosage form 10 of FIGS. 32 and 33 operate as described above with the added embodiment that all of film 18 can be used for increasing the volume of compartment 14, and that all of film 18 can be used for decreasing the volume of compartment 13, thereby insuring the extended, continuous and constant release of proton pump inhibitor 16 from compartment 13 to the exterior of dosage form 10.

In another embodiment, the invention is represented by reference to FIGS. 34-36. If necessary to prevent the release, and subsequent degradation, of the proton pump inhibitor into the acidic stomach, the dosage form 10 may be coated with an enteric coating layer or comprise an enteric polymer as described, for example, in FIGS. 37-41.

The invention relates to a dosage form for delivering proton pump inhibitor at a substantially constant rate over time. The dosage form comprises (1) a wall formed of a microporous polymer, or (2) a wall formed in part of a microporous polymer with the remaining part of the wall formed of a semipermeable polymer. The wall in (1) surrounds a compartment comprising a flexible partition that separates the compartment into a first space containing the beneficial proton pump inhibitor and a second space containing a swellable polymer. The wall in (2) surrounds a compartment comprising a flexible partition that separates the compartment into a first space in contact with the microporous wall and containing the proton pump inhibitor, and a second space in contact with the semipermeable polymer containing an osmotically effective solute, or a swellable polymer. In operation, the proton pump inhibitor is delivered from the dosage form by (a) fluid diffusing through the microporous wall into the second space causing the polymer to swell, or by (b) fluid being imbibed through the semipermeable wall into the second space causing the solute to dissolve and form a solution, or causing the polymer to swell, wherein in (a) or (b), the second space expands against the partition urging it to move into the first space and maintain the proton pump inhibitor in a saturated state at the microporous wall, with the proton pump inhibitor diffusing from the first space through fluid filled micropaths in the wall from the dosage form at a substantially zero order rate over an extended period of time.

In FIG. 34, dosage form 10 comprises a body 11, that is shaped, sized, structured and adapted for easy placement and prolonged retention in an environment of use for the extended, continuous delivery of proton pump inhibitor thereto.

In FIG. 35 dosage form 10 of FIG. 36 is seen in opened-section with a part of its outer layer removed for elucidating the total structure of dosage form 10. In FIG. 35, dosage form 10 comprises a body 11 having an exterior wall 12 that surrounds an internal space having a partition 14 that separates the space into a first space 15 and a second space 16. Space 16 contains a swellable polymer 18, that on swelling in the presence of water exerts pressure on partition 14 causing it to move and occupy volume in space 15. The actions of partition 14 and polymer 18 combine to decrease the volume of space 15, thereby functioning to maintain beneficial agent 17 in a saturated state in space 15, especially during the time dosage form 10 is in operation in a prechosen environment of use.

In FIG. 35, wall 12 of dosage form 10 is formed of a microporous polymeric material containing a plurality of microscopic-sized interconnected pores or voids. The pores, illustrated as circles 13 for discussion herein, can be continuous with openings on both sides of wall 12, the pores can be interconnected through tortuous paths of regular and irregular shapes, including curved, curved-linear, randomly oriented continuous paths, hindered connected paths and pores, and other paths and pores discernible by microscopic examination. Generally, materials possessing from 5 to 95% pores, more preferably a void space of 30% to 90%, and having a pore size of 100 angstroms to 200 microns can be used for making wall 12. The pores and connecting intra-wall paths can be preformed in the polymer, which microporous polymer is then manufactured as wall 12 of system 10. Materials useful for making the microporous wall 12 include polycarbonates comprised of linear polyesters of carbonic acid in which carbonate groups recur in the polymer chain, microporous materials prepared by the phosgenation of a dihydroxyl aromatic such as a bisphenol A, microporous poly(vinylchloride), microporous polyamides such as polyhexamethylene adipamide, microporous modacrylic copolymers including those formed from poly(vinylchloride) 60% and acrylonitrite, microporous styrene-acrylic copolymers, porous polysulfones characterized by diphenylene sulfone groups in a linear chain thereof, halogenated poly(vinylidene), polychloroethers, acetal polymers, polyesters prepared by esterification of a dicarboxylic acid or anhydride with an alkylene polyol, poly(alkylenesulfides), phenolic polyesters, microporous poly(saccharides), microporous poly(saccharides) having substituted anhydroglucose units exhibiting a decrease permeability to the passage of water and biological fluids, asymmetric porous polymers, cross-linked microporous olefin polymers, hydrophobic or hydrophilic microporous homopolymers, copolymers having a reduced bulk density, and materials described in U.S. Pat. Nos. 3,595,752; 3,643,178; 3,654,066; 3,709,774; 3,718,532; 3,803,061; 3,852,224; 3,852,388; and 3,853,601, in British Pat. No. 1,126,849, and in Chem. Abst., Vol. 71 427F, 22573F, 1969. In another embodiment, wall 12 contains a multiplicity of pore-formers, not shown, that are dissolved or leached from wall 12, which is integrally manufactured as dosage form 10. The pore-formers suitable for the invention include solids having a size of about 100 angstroms to 200 microns, and they include alkali metal salts such as lithium carbonate, sodium chloride, sodium bromide, sodium carbonate, potassium chloride, potassium sulfate, potassium phosphate, sodium benzoate, sodium acetate, sodium citrate, potassium nitrite, and the like. The alkaline earth metal salts such as calcium phosphate, calcium nitrate, calcium chloride, and the like. The transition metal salts such as ferric chloride, ferrous sulfate, zinc sulfate, cupric chloride, manganese fluoride, manganese fluorosilicate, and the like. Organic compounds such as polysaccharides including pentoses, hexoses, disaccharides, sugars, sucrose, glucose, fructose, mannitol, mannose, galactose, aldohexose, altrose, talose, sorbitol, and the like, carboxy-polymethylene, Carbowax® compounds, polysorbate, and the like. In this embodiment, the pore-formers are removed when dosage form 10 is in the environment of use, thereby forming microporous wall 12 in the environment. Additional micropores materials for forming wall 12 include microporous poly(urethanes), cross-linked, chain-extended microporous poly(urethanes), microporous poly(urethanes) in U.S. Pat. No. 3,524,753, microporous poly(imides), microporous poly(benzimidazoles), regenerated microporous proteins, semi-solid cross-linked microporous poly(vinylpyrrolidone), microporous materials prepared by diffusion of multivalent cations into polyelectrolyte sols as in U.S. Pat. No. 3,565,259, anisotropic permeable microporous materials of ionically associated polyelectrolytes, microporous polymers formed by the coprecipitation of a polycation and a polyanion as described in U.S. Pat. Nos. 3,276,589; 3,541,006; 3,541,055; and 3,546,142, microporous derivatives of poly(styrene) such as microporous poly(sodium styrene-sulfonate) and microporous poly(vinyl benzyltrimethyl-ammonium chloride), the microporous materials disclosed in U.S. Pat. No. 3,615,024 and U.S. Pat. Nos. 3,646,178 and 3,852,224.

The microporous paths of wall 12 are prefilled or filled in the environment of use with a diffusive medium permeable to the passage of proton pump inhibitor 17. The medium is generally non-toxic and it does not adversely affect the system, the wall, the proton pump inhibitor and the environment. In one embodiment, the medium is a liquid phase comprised of a solution, a colloidal medium, or a sol, the medium can be polar, semi-polar or non-polar, or it can be a liquid present in the environment of use, including water, biological fluids, saline, and buffers.

Partition 14 of dosage form 10 consists, in one embodiment, of a film made of a semipermeable polymer that is essentially impermeable to the passage of proton pump inhibitor, osmotic solute and polymer and is permeable to the passage of fluid that enters dosage form 10; and, in another embodiment partition 14 is made of a film impermeable to proton pump inhibitor, solutes, polymers and fluid. The semipermeable polymers include cellulose acrylate, cellulose diacylate, cellulose triacylate, cellulose ethers and cellulose esters. Typical semipermeable polymers include cellulose acetate, cellulose acetate ethyl carbamate, and the like. Other semipermeable polymers include polyurethane, and selectively permeable polymers formed by the coprecipitation of a polyanion and a polycation, and semipermeable ion exchange polymers. Exemplary polymers suitable for partition 14, when it is impermeable to fluid agents and solutes include, plasticized polyvinyl chloride, styrene-butadiene block copolymer, polyester-polyethers, ethylene-propylene copolymer, segmented block polyurethane, chlorinated polyethylene, ethylene vinylchloride copolymer, and the like. Partition 14 is suitably joined to wall 12 during manufacture of dosage form 10, and can contain a plasticizer that imparts flexibility and expandability to partition 14. Exemplary plasticizers suitable for adding to partition 14 to impart flexibility and stretchability include cyclic and acyclic plasticizers. Typical plasticizers are those selected from the group consisting of phthalates, phosphates, citrates, adipates, tartrates, sebacates, succinates, glycolates, glycerolates, benzoates, myristates, sulfonamides, halogenated phenyls, glycols, diols, and polyols. Exemplary plasticizers further include dialkyl phthalates, dicycloalkyl phthalates, diaryl phthalates and mixed alkyl-aryl phthaltes as represented by dimethyl phthalate, dipropyl phthalate, di(2-ethylhexyl)-phthalate, di-isopropyl phthalate, diamyl phthalate and dicapryl phthalate; alkyl and aryl phosphates such as tributyl phosphate, trioctyl phosphate, tricresyl phosphate, trioctyl phosphate, tricresyl phosphate and triphenyl phosphate; trieresyl phosphate, trioctyl phosphate, tricresyl phosphate and triphenyl phosphate; alkyl and aryl phosphates such as tributyl phosphate, trioctyl phosphate, tricresyl phosphate, trioctyl phosphate, tricresyl phosphate and triphenyl phosphate; alkyl citrate and citrates esters such as tributyl citrate, triethyl citrate, and acetyl triethyl citrate; alkyl adipates such as dioctyl adipate, diethyl adipate and di(2-methoxyethyl)-adipate; dialkyl tartrates such as diethyl tartrates and dibutyl tartrate; alkyl sebacates such as diethyl sebacate, dipropyl sebacate and dinonyl sebacate; alkyl succinates such as diethyl succinate and dibutyl succinate; alkyl glycolates, alkyl glycerolates, glycol esters and glycerol esters such as glycerol diacetate, glycerol triacetate, glycerol monolactate diacetate, methyl phythayl ethyl glycolate, butyl phthalyl butyl glycolate, ethylene glycol diacetate, triethylene glycol dibutyrate and triethylene glycol dipropionate. Other plasticizers include camphor, N-ethyl-(o- and p-toluene) sulfonamide, chlorinated biphenyl, benzophenone, N-cyclohexyl-ptoluene sulfonamide, substituted epoxides, poly(alkylene glycols), poly(alkylene diols), esters of alkylene glycols, and the like. In operation, when compartment 16 contains polymer 18, the polymer 18 absorbs fluid that enters compartment 16 causing 18 to swell, expand and fill compartment 16, and also, swell and expand against partition 14, causing it to move and occupy the space of compartment 15. This action correspondingly reduces the amount of space available for proton pump inhibitor 17, and this continual decrease in space substantially keeps proton pump inhibitor 17 in a substantially saturated phase.

Dosage form 10 of FIG. 36 is similar to dosage form 10 of FIG. 35, with dosage form 10 of FIG. 36 embracing other structural embodiments. The embodiments of FIG. 36 include wall 12 having at least one surface 12a formed of a semipermeable polymer. When wall 12a is formed of a semipermeable polymer, space 16 contains a member selected from the group consisting essentially of an osmotically effective solute and a swellable polymer 18. Osmotically effective compounds useful for this purpose include magnesium sulfate, magnesium chloride, sodium chloride, lithium chloride, potassium sulfate, sodium carbonate, potassium acid phosphate, mannitol, urea, sucrose, and the like. The osmotically effective compounds are also known as osmagents and they are disclosed in U.S. Pat. Nos. 3,854,770 and 4,077,407. Swellable polymers that be used in space 16 are cross-linked hydrogels including poly(hydroxyalkylmethacrylates), poly(acrylamide), poly(methacrylamide), poly(N-vinyl-2-pyrrolidone), anionic and cationic hydrogels, polyelectrolyte complexes, a water-insoluble, water-swellable copolymer produced by forming a dispersion of finely divided copolymers of maleic anhydride with styrene, ethylene, propylene, butylene or isobutylene cross-linked with from about 0.001 to about 0.5 moles of a polyunsaturated cross-linking agent per mole of maleic anhydride in the copolymer as disclosed in U.S. Pat. No. 3,989,586, the water-swellable polymers of N-vinyl lactams as disclosed in U.S. Pat. No. 3,992,562, semi-solid cross-linked poly(vinyl pyrrolidone), diester cross-linked polyglucan hydrogels as described in U.S. Pat. No. 4,002,173, the anionic hydrogels of heterocyclic N-vinyl monomers as disclosed in U.S. Pat. No. 4,036,788, the ionogenic hydrophilic gels as described in J. Biomedical Mater. Res., Vol. 7, pages 123 to 126, 1973, and the like. When space 16 houses the solute or the swellable polymer, partition 14 is formed of a member selected from the group consisting of a semipermeable polymer, and an impermeable polymer. When space 16 contains an osmotic solute, in operation it imbibes fluid through semipermeable wall 12a in a tendency towards osmotic equilibrium, to dissolve the solute and form a solution that fills space 16, apply pressure against partition 14, urging it to move into space 15 and decrease its volume, thereby keeping the proton pump inhibitor present at the microporous wall 12. When space 16 contains a swellable polymer, it absorbs fluid, expands, but does not dissolve in fluid that enters space 16. The expanding polymer pushes against partition 14 causing it to move into space 15, thereby keeping proton pump inhibitor 17 in a saturated state at the release rate wall.

The wall forming the dosage form of the invention is a material that is semi-permeable, for example a material that is permeable to an external fluid such as water and the like while essentially impermeable to the proton pump inhibitor composition in the dosage form. The material forming the wall can be non-erodible or bioerodible after a predetermined extended period of time and in each instance it is semi-permeable to solvent but not to solute and is suitable for construction of the osmotic powered dosage form. Typical materials for forming the wall include membranes known to the art as osmosis and reverse osmosis membranes such as commercially available unplasticized cellulose acetate, plasticized cellulose acetate, reinforced cellulose acetate, cellulose nitrate with 11 percent nitrogen, cellulose diacetate, cellulose triacetate, agar acetate, amylose triacetate, beta glucan acetate, beta glucan triacetate, cellulose acetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxylated ethylene-vinylacetate, cellulose acetate butyrate having a viscosity of from about 10 seconds to about 50 seconds, cellulose acetate butyrate containing about 17 percent of combined butyryl and about 29.5 percent acetyl, permselective, aromatic nitrogen-containing polymeric membranes that exhibit water permeability and essentially no solute passage, osmosis membranes made from polymeric epoxides, osmosis membranes made from copolymers of an alkylene oxide and alkyl glycidyl ether, semi-permeable polyurethanes, semi-permeable polyglycolic or polylactic acid and derivatives thereof, thin film membranes are disclosed in U.S. Pat. No. 3,133,132, the membranes of ionically associated polyelectrolytes, the polymers formed by the coprecipitation of polycation and a polyanion as described in U.S. Pat. Nos. 3,276,586, 3,541,005, 3,541,006, 3,546,142, 3,173,876; derivatives of polystyrene such as poly(sodium styrenesulfonate) and poly(vinylbenzyltrimethyl-ammonium chloride), and the like. Generally, membranes having a fluid permeability of 0.01 to 10 cc/cm²/hour or day/or higher at atmosphere pressure against a saturated product solution or saturated solute solution to a changing concentration at the temperature of use while simultaneously possessing a high degree of impermeability to the product or solute are useful and within the spirit of the invention.

Various osmotically effective solutes including organic and inorganic compounds are advantageously used when it is desired to release a composition comprising at least one proton pump inhibitor from the dosage form. The osmotically effective compounds or solutes confined in the dosage form are a substantial motive force of the dosage form and they exhibit an osmotic pressure gradient against an external fluid across the membrane while the membrane is substantially impermeable to the passage of the osmotically effective solute to prevent loss thereof through the membrane. The solutes are conveniently used by dispensing or homogenously or heterogeneously mixing a solute or a mixture of solutes with the composition comprising the proton pump inhibitor either before they are charged into the compartment or by self mixing after charging a solute and composition into the compartment. In operation, these solutes osmotically attract fluid into the dosage form to produce a solution of the solute which is released from the dosage form concomitantly transporting therewith undissolved and dissolved composition comprising the proton pump inhibitor. Various osmotically effective solutes include compounds such as magnesium sulfate, magnesium chloride, sodium chloride, lithium chloride, potassium sulfate, sodium carbonate, sodium sulfite, lithium sulfate, calcium bicarbonate, sodium sulfate, calcium sulfate, potassium acid phosphate, calcium lactate, magnesium succinate, tartaric acid, soluble carbohydrates such as raffinose, glucose, mixtures thereof and the like. The solid solute, present initially in excess, can be in any suitable physical form such as particles, crystals, pellets, tablets, strips, film, granules and the like.

Additionally, the composition and composition solute can be used in a mixed form by mixing the composition or product with a binder. The product in powdered, granular, piece and the like form, is homogenously or heterogeneously dispersed in the binder which binder is water soluble or water insoluble but will release the product on contact with water. Typical water soluble binders include polyethylene glycol, gelatin, agar, carboxycellulose, ethylmethylcellulose, polyvinyl alcohol, polyvinylpyrrolidone, water soluble starch derivatives and the like. Typical water insoluble binders that can comprise about 1 to 50 percent of the composition include cellulose acetate, polyurethane, epoxides, and other insoluble binders that permit the free movement of water into the pores of the structure to transport the product from the binder.

The amount of composition present in the dosage form, whether soluble, a derivatized soluble form thereof, is generally non-limited and it is an amount larger than or equal to the amount of the composition that is necessary to osmotically operate the dosage form and on its release from the dosage form is effective for bringing about the product's desired effect. Since the invention contemplates a variety of dosage forms of various sizes and shapes, for a variety of uses, there is no critical upper limit on the amount of product incorporated in the dosage form. The lower limit will depend on osmotic activity, the span of the release of the product and the activity of the product. Generally, the dosage form will contain about 0.01 percent to 90 percent or higher of a proton pump inhibitor composition or a mixture of proton pump inhibitor composition and solute based on the weight of the proton pump inhibitor composition or solute to the volume of the dosage form.

The expressions “passageway” and “passageway communicating with” are defined herein and methods for making them are described herein. Passageways are also referenced in FIGS. 37-41.

The dissolution of a drug indicates the drug entering into solution upon its delivery from a dosage form provided by the embodiments of the invention is measured by the following procedure. First, a drug receiving solution, such as, intestinal fluid, is used as the dissolution media. A dosage form prepared by the invention is placed into the dissolution media and the proton pump inhibitor released by the dosage form into the dissolution media is sampled at a constant time interval over the time period of dissolution. The filtered samples are assayed by a reversed high pressure liquid chromatography, or detection by UV. The concentration of the samples is measured against a standard curve containing, for example, at least five standard points. Procedures for dissolution testing are reported in The United States Pharmacopoeia, The National Formulary, pp. 1791 to 1796; (1995); Pharmaceutical Sciences, by Remington, 17.sup.th Ed., pp. 653-666 (1985); and USP XXII, Dissolution Paddle Analysis, pp. 1578-1579 (1990).

The release rate of proton pump inhibitor from a dosage form manufactured by the invention can be ascertained by the following procedure. The procedure comprises placing the dosage form in a solution, usually water, and taking aliquots of the release rate solution, followed by their injection into a chromatographic system to quantify the amount of proton pump inhibitor released during specified test intervals. The proton pump inhibitor, for example, is resolved on a column and detected by UV absorption. Quantitation is performed by linear regression analysis of peak areas from a standard curve containing at least five standard points.

The release rate procedure comprises attaching a dosage form to a plastic rod with the orifice exposed to the drug receiving solution. Then, attaching the rod to a release arm, with the arm affixed to an up/down reciprocating shaker, which operates at amplitude of about 3 cm and 2 seconds per cycle. Then, continuously immersing the dosage form in 50 ml test tubes containing 30 ml of H₂O, equilibrated in a constant temperature water bath at 37° C.±0.5° C. Next, at the end of each interval, transfer the dosage form to the next row of new test tubes containing a receiving solution, such as water. After the release pattern is complete, remove the tubes and allow to cool to room temperature, followed by filling the calibrated tubes to the 50 ml mark with a solvent, such as acetone. The samples are mixed immediately, transferred to sample vials, followed by chromatography analysis.

Any proton pump inhibitor known in the art can be used in the compositions and dosage forms described herein. Exemplary proton pump inhibitors include rabeprazole, omeprazole, lansoprazole, esomeprazole, pantoprazole, leminoprazole, timoprazole, tenatoprazole, disuiprazole, RO 18-5362, IY 81149, 3-butyl-4-(2-methylphenylamino)-8-(2-hydroxyethoxy)-quinoline, and the like. Among these, rabeprazole, lansoprazole, esomeprazole, pantoprazole, leminoprazole, timoprazole, tenatoprazole, disulprazole, RO 18-5362, IY 81149, and 3-butyl-4-(2-methylphenylamino)-8-(2-hydroxyethoxy)-quinoline are preferred.

In one embodiment, the proton pump inhibitors are compounds of formula (1), pharmaceutically acceptable salts thereof, and/or stereoisomers thereof:

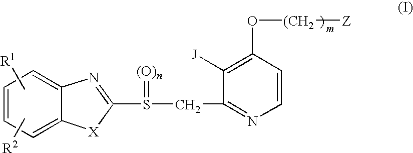

wherein R¹ and R² are each independently a hydrogen atom, a halogen atom, a lower alkyl, lower alkoxy, halogenated lower alkyl, lower alkoxycarbonyl or carboxyl group;

X is —O—, —S— or ═N—R³, wherein R³ is a hydrogen atom or a lower alkyl, phenyl, benzyl or lower alkoxycarbonyl group; and

Z is:

1. —O(CH₂)ₚ—O—R⁴
wherein p is an integer of 1 to 3 and R⁴ is hydrogen atom or a lower alkyl, aryl or aralkyl group,

2. —O—(CH₂)_{q}—R⁵
wherein q is an integer of 1 to 3 and R⁵ is a halogen atom or an alkoxycarbonyl, aryl or heteroaryl group,

3. —O—(CH₂)ᵣO—(CH₂)ₛ—O—R⁶
wherein r and s are each independently an integer of 1 to 5 and R⁶ is a hydrogen atom or a lower alkyl group,

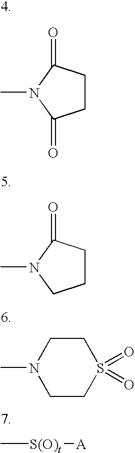

wherein t is an integer of 0 to 2, and A is a lower alkyl, alkoxycarbonylmethyl, pyridyl, furyl,

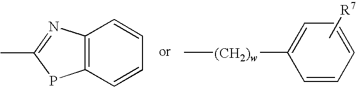

wherein B is —NH—, —O— or —S—, and w is an integer of 0 or 1;

8. —N(R⁸)—CH₂—C₆H₅
wherein R⁸ is an acetoxy or lower alkyl group;

9. —OR⁹
wherein R⁹ is a hydrogen atom, a lower alkyl or aryl group;
   n is an integer of 0 to 2; m is an integer of 2 to 10, and J and K are each independently a hydrogen atom or a lower alkyl group, with the proviso that when Z is a group falling under the above category (9), then R⁹ is a lower alkyl group and m stands for an integer of 3 to 10, and pharmaceutically acceptable salts thereof.

The same definitions for R¹, R², X, n, J, K, Z and m are used throughout the specification that follows and in the appended claims.

In the definition of the compounds of formula (I), the lower alkyl group defined with respect to R¹, R², R³, R⁴, R⁶, R⁷, R⁸, A, J and K can be a straight-chain or branched alkyl group having 1 to 6 carbon atoms. Examples include methyl, ethyl, n-propyl, n-butyl, isopropyl, isobutyl, 1-methylpropyl, tert-butyl, n-pentyl, 1-ethylpropyl, isoamyl and n-hexyl groups, among which methyl and ethyl groups are most preferred.

The lower alkoxy group and the lower alkoxy moiety of the lower alkoxycarbonyl group defined above with respect to R¹ and R² can be an alkoxy group derived form the above lower alkyl group. Methoxy and ethoxy groups are most preferred.

The halogen atom defined above includes chlorine, bromine, iodine or fluorine. The aryl group defined above with respect to R⁴ and R⁵ can be phenyl, tolyl, xylyl, napthyl or the like, which can be substituted with a lower alkoxy or hydroxyl group, a halogen atom or the like.

Examples of the arylalkyl defined above with respect to R⁴ include benzyl and phenethyl groups.

Examples of the heteroaryl group defined above with respect to R⁵ include pyridyl and furyl groups.

In the definition of Z in formula (I), groups 1, 2, 3, 4, 5 and 9 are preferred; and group 9 is the most preferred. As for R¹ and R², hydrogens for both and then a combination of a lower alkyl (e.g., methyl) for R¹ and hydrogen for R² are preferred. X is preferably ═NR³, where R³ is hydrogen. A preferred value for n is 1. The preferred substituents for J and K are both hydrogen or where J is lower alkyl (e.g., methyl), and K is hydrogen, or when J is hydrogen and K is lower alkyl (e.g., methyl). Thus, J or K are independently preferably hydrogen, or methyl, most preferably J is methyl and K is hydrogen.

In another embodiment, the compounds of formula (I) are compounds of formula (A), pharmaceutically acceptable salts thereof, and/or stereoisomers thereof:

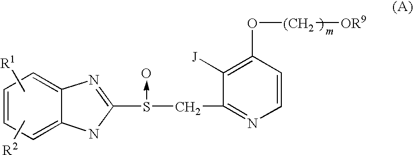

wherein R¹, R², J, m and R⁹ have the same meanings as defined above.

In formula (A), the preferred R¹ and R² substituents are both hydrogen, or R¹ is 5-lower alkoxy, 5-lower alkyl or 5-halogenated lower alkyl and R² is hydrogen. The preferred substituent for J is hydrogen or methyl; the preferred value for m is in the range of 3 to 10, the most preferred being 3; and the preferred R⁹ substituent is lower alkyl (e.g., methyl), or aryl. Among these possibilities for the compounds of formula (A), the preferred combination is when R¹ and R² are both hydrogen, J is methyl, m is 3 and R⁹ is methyl.

Another group of preferred compounds in formula (A) are combinations of the above substituents where both R¹ and R² are hydrogen, J is hydrogen, m is 3 and R⁹ is methyl.

Another group of preferred compounds falling within formula (A) is when both R¹ and R² are hydrogen, J is methyl, m is 2 and R⁹ is benzyl.

In another embodiment, the compounds of formula (I) are compounds of formula (B), pharmaceutically acceptable salts thereof, and/or stereoisomers thereof:

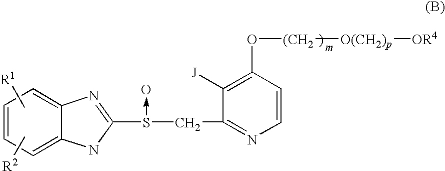

wherein R¹, R², J, p, m and R⁴ have the same meanings as given above.

In formula (B), the preferred substituents for R¹ and R² are both hydrogen; or when R¹ is 5-lower alkoxy, 5-lower alkyl or 5-halogenated lower alkyl, R² is hydrogen. The preferred value of m is 2 or 3; the preferred value for p is 2 or 3; and the preferred substituent for R⁴ is methyl or benzyl. Of the above possibilities for formula (B), the most preferred combination is where R¹ is 5-methyl, R² is hydrogen, J is methyl, m is 2, p is 2 and R⁴ is methyl.

In another embodiment, the compound of formula I is a compound of formula (C), a pharmaceutically acceptable salt thereof, and/or a stereoisomer thereof:

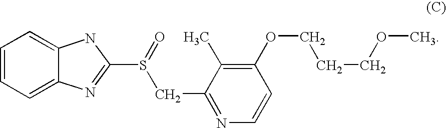

Preferably, the compound of formula (C) is a sodium salt, which is known as rabeprazole sodium or ACIPHEX® (Eisai Inc., Teaneck, N.J.).

Although the compounds of the invention can be present as a hydrate or as a stereoisomer, the hydrates and stereoisomers are included within the scope of the invention. For example, the compound of formula (C) can be a compound of formula (D) or a pharmaceutically acceptable salt thereof (e.g., a sodium salt):

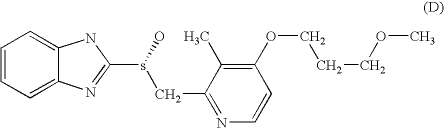

The compound of formula (I)) is R (+) rabeprazole.

Alternatively, the compound of formula (C) can be a compound of formula (E) or a pharmaceutically acceptable salt thereof (e.g., a sodium salt):

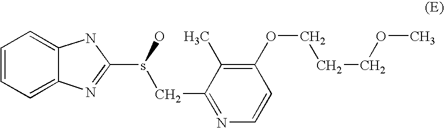

The compound of formula (E) is S (−) rabeprazole.

The compounds of the invention can be administered as any pharmaceutically acceptable salt known in the art. Pharmaceutically acceptable salts are known in the art and include those of inorganic acids, such as hydrochloride, sulfate, hydrobromide, sulfate, and phosphate; those of organic acids, such as formate, acetate, maleate, tartrate, trifluoroacetate, methanesulfonate, benzenesulfonate and toluenesulfonate, and those of amino acids such as arginine, aspartic acid and glutamic acid. When certain substituents are selected, the compounds of the invention can form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylenediamine. One skilled in the art will recognize that the compounds of the invention can be made in the form of any of these or of any other pharmaceutically acceptable salt. For example, compounds represented by formula (I), wherein X is ═N—R³ and R³ is a hydrogen atom, or compounds represented by formula (I), wherein Z is a group falling under the category 7 and B is a group of —NH—, can be present as a metal salt, such as sodium, potassium, magnesium or calcium.

The proton pump inhibitors are commercially available or can be prepared by processes known in the art. Rabeprazole sodium is commercially available as ACIPHEX® from Eisai Inc., Teaneck, N.J., and is described, for example, in U.S. Pat. No. 5,045,552, the disclosure of which is incorporated by reference herein in its entirety. Methods for preparing R (+) rabeprazole are described in WO 99/55157, the disclosure of which is incorporated by reference herein in its entirety. Methods for preparing S (−) rabeprazole are described in WO 99/55158, the disclosure of which is incorporated by reference herein in its entirety.

The proton pump inhibitors can be administered in amounts of about 0.001 to about 500 mg per day; from about 0.01 mg to about 400 mg per day; from about 0.01 mg to about 300 mg per day; from about 0.01 to about 200 mg per day; preferably from about 0.05 to about 100 mg per day; preferably about 0.05 to about 50 mg per day, more preferably about 0.1 to about 40 mg per day, still more preferably about 10 to about 30 mg per day, still more preferably about 10 to about 20 mg per day. The proton pump inhibitors can be administered once a day or in divided doses, for example from 2 to 4 times a day, preferably once per day. One skilled in the art will recognize that when the compounds and/or compositions of the invention are administered to infants or children, the dose can be smaller than the dose administered to adults, and that the dose can be dependent upon the size and weight of the patient.

The invention provides methods for treating gastrointestinal disorders using the extended release compositions. The gastrointestinal disorder can be any in the art, and can include gastrointestinal disorders of the upper and/or lower gastrointestinal tracts. Exemplary gastrointestinal disorders include ulcers, post-operative aspiration, dyspepsia, acute gastrointestinal bleeding, lower esophageal mucosal rings, esophageal strictures, esophageal dismotility, hiatal hernia, achlasia, irritable bowel syndrome, Barrett's esophagus, gastroparesis, gastrointestinal motility disorders, diverticulosis, diverticulitis, malabsorption syndromes, gastroesophageal reflux disease (GERD), problems caused by esophageal bypass surgery, belching, eructation, flatulence, diarrhea, inflammatory bowel disease, infectious enteritis, idiopathic gastric acid hypersecretion, gastritis, constipation, colic, vomiting, nausea, motion sickness, gastrointestinal injuries, esophageal injuries, gastric mucosal injuries, short bowel syndrome, bowel dysfunctions, early satiety, abdominal pain, abdominal bloating, sour stomach, radiation-induced injury to the gastrointestinal tract, gastrointestinal disorders induced by medications, chronic sore throat, noncardiac chest pains, coughing, dysphagia, Shwachman syndrome, decreased gastric mucin production, iron deficiency anemia, decreased nasal airflow, pancreatitis, cystic fibrosis and the like.

“Ulcers” include peptic ulcers, bleeding peptic ulcers, stress ulcers, stomal ulcers, refractory ulcers, ICU-induced ulcers, esophageal ulcers, Zollinger-Ellison syndrome, post-operative ulcers and the like. “Peptic ulcers” include gastric ulcers and duodenal ulcers. The ulcers can be associated with H. pylori.

The invention is particularly suited to the administration of proton pump inhibitors against Helicobacter pylori which are able to penetrate the space between the inner stomach lining and the stomach protective mucous layer, where the Helicobacter pylori organism is present, with the result of eradicating the Helicobacter pylori organism either totally or to such a degree that relapse after treatment for a large portion of the treatment population is minimized. The increased residence time of the proton pump inhibitor in the stomach provided by this invention permits a proton pump inhibitor delivery period at the situs of the organism that is longer than that provided by conventional tablets and capsules. The increased efficiency and efficacy of treatment afforded by the invention allows one to treat gastric disorders in a large number of subjects with dosage forms having a single proton pump inhibitor. Accordingly, one avoids the necessity of having to employ complicated treatment regimens directed to the elimination of the Helicobacter pylori organism, such as those that use multiple drug regimens.

Each of the patents and publications cited herein are incorporated by reference herein in their entirety.

It will be apparent to one skilled in the art that various modifications can be made to the invention without departing from the spirit or scope of the appended claims.

What is claimed is:

1. A pharmaceutical composition comprising a therapeutically effective amount of a proton pump inhibitor, a polymer and a hydrogel.

2. A pharmaceutical composition comprising a semipermeable wall that forms a compartment, wherein the compartment comprises a therapeutically effective amount of a proton pump inhibitor and an osmotically effective solute; wherein the semipermeable wall comprises at least one passageway.

3. A pharmaceutical composition comprising (i) a core which comprises a therapeutically effective amount of a proton pump inhibitor and a pharmaceutically acceptable carrier; (ii) a first coat that surrounds the core, wherein the first coat comprises at least one polymer; (iii) a second coat that surrounds the first coat, wherein the second coat is permeable to the passage of fluid and impermeable to the passage of proton pump inhibitor; and (iv) a passageway in the first and second coats for releasing the proton pump inhibitor from the core.

4. The pharmaceutical composition of claim 3, wherein the first coat comprises ethyl cellulose, hydroxylalkylcellulose, or a mixture thereof.

5. A pharmaceutical composition comprising (i) a therapeutically effective amount of a proton pump inhibitor; (ii) a polymer matrix; and (iii) at least one band of an insoluble material circumscribing at least a portion of the surface of the polymer matrix.

6. The pharmaceutical composition of claim 5, wherein the proton pump inhibitor is dispersed or dissolved in the polymer matrix.

7. The pharmaceutical composition of claim 5, wherein the polymer matrix comprises at least one water-soluble polymer and at least one hydroattractant.

8. The pharmaceutical composition of claim 5, further comprising at least one gastric-emptying delaying agent.

9. A pharmaceutical composition comprising (a) a wall that is permeable to the passage of fluid and is substantially impermeable to the passage of proton pump inhibitor, which wall surrounds and forms; (b) a compartment comprising (i) a first composition which comprises a therapeutically effective amount of a proton pump inhibitor; at least one osmopolymer; and, optionally, at least one osmagent; and (ii) a second composition in contact with the first composition in the compartment, wherein the second composition, in the presence of fluid, increases in dimension and pushes the drug composition out of the pharmaceutical composition; and (d) at least one exit means in the wall for delivering the first composition from the pharmaceutical composition.

10. A pharmaceutical dosage form comprising: (a) a wall that is permeable to the passage of an exterior fluid present in the environment of use and substantially impermeable to the passage of proton pump inhibitor; wherein the wall surrounds and forms: (b) a compartment comprising (i) a first composition comprising a therapeutically effective amount of a proton pump inhibitor, an osmopolymer that exhibits an osmotic pressure gradient across the wall against an external fluid, and, optionally, an osmagent that exhibits an osmotic pressure gradient across the wall against an external fluid; and (ii) a second composition comprising an osmopolymer that exhibits an osmotic pressure gradient across the wall against an external fluid, and, optionally, an osmagent that exhibits an osmotic pressure gradient across the wall against an external fluid; and (c) at least one passageway in the wall communicating with the first composition and the exterior of the dosage form for delivering the proton pump inhibitor through the passageway from the dosage form.

11. The pharmaceutical dosage form of claim 10, wherein the first composition is in the compartment as a layer, and the second composition is in the compartment as a separate layer.

12. The pharmaceutical dosage form of claim 10, wherein the osmopolymer comprising the second composition has a molecular weight greater than the molecular weight of the osmopolymer comprising the first composition.

13. A pharmaceutical dosage form comprising: (a) a wall permeable to the passage of an exterior biological fluid and substantially impermeable to the passage of proton pump inhibitor formulation, which wall surrounds and forms: (b) a compartment comprising: (1) a drug formulation which comprises a therapeutically effective amount of a proton pump inhibitor, an osmotically effective solute that is soluble in the exterior fluid and exhibits an osmotic pressure gradient across the wall against the fluid and a polymer that imbibes fluid and absorbs fluid that enters the compartment; and (2) a delivery formulation which comprises an osmotically effective solute that is soluble in the exterior fluid and exhibits an osmotic pressure gradient across the wall against the fluid and a polymer that imbibes fluid and absorbs fluid that enters the compartment; and (c) at least one passageway in the wall connecting the exterior of the dosage form with the drug formulation for delivering the drug formulation from the dosage form to the exterior environment.

14. A pharmaceutical composition comprising in combination: (1) a first composition comprising a therapeutically effective amount of a proton pump inhibitor, an osmopolymer, and, optionally, an osmagent; and (2) a second composition in laminar arrangement with the first composition, wherein the second composition comprises an osmopolymer and, optionally, an osmagent; wherein the first and second compositions exhibit an osmotic pressure gradient across a semipermeable polymeric film against fluid.

15. An osmotic delivery system comprising: (a) a semipermeable wall permeable to the passage of fluid and substantially impermeable to the passage of a proton pump inhibitor, wherein the semi-permeable wall surrounds and forms; (b) a compartment comprising a therapeutically effective amount of a proton pump inhibitor; and (c) an osmotic passageway in the wall communicating with the compartment and the exterior of the system for releasing the proton pump inhibitor through the osmotic passageway to the exterior of the osmotic delivery system.

16. The osmotic delivery system of claim 15, wherein the semipermeable wall comprises an organic solvent soluble polymer and/or a permeability enhancer.

17. The osmotic delivery system of claim 15, wherein the semipermeable wall comprises an organic solvent soluble polymer and a blend of water soluble polymers which, on the application of energy used to coat the semipermeable wall of the delivery system, form a hydrophilic and substantially fluid insoluble polymer in the semipermeable wall.

18. The osmotic delivery system of claim 15, wherein the semipermeable wall comprises an organic solvent soluble polymer, a polyhydroxy polymer and a polycarboxy polymer, wherein the polyhydroxy polymer and polycarboxy polymer react while coating the wall to form a hydrophilic fluid permeability enhancing polymer blended within the organic solvent soluble polymer.

19. The composition, dosage form or system of claim 1, 2, 3, 5, 9, 10, 13, 14 or 15, wherein the proton pump inhibitor is selected from the group consisting of omeprazole, lansoprazole, esomeprazole, pantoprazole, leminoprazole, timoprazole, tenatoprazole, disulprazole, RO 18-5362, IY 81149, 3-butyl-4-(2-methylphenylamino)-8-(2-hydroxyethoxy)-quinoline, a pharmaceutically acceptable salt thereof and/or a stereoisomer thereof.

20. The composition, dosage form or system of claim 1, 2, 3, 5, 9, 10, 13, 14 or 15, wherein the proton pump inhibitor is rabeprazole, a pharmaceutically acceptable salt thereof and/or a stereoisomer thereof.

21. The composition, dosage form or system of claim 1, 2, 3, 5, 9, 10, 13, 14 or 15, further comprising an enteric polymer in an amount sufficient to prevent release of the proton pump inhibitor in the patient's stomach and to allow release of the proton pump inhibitor in the patient's intestine.
